SEQUENCE LISTING (19 sequences)

SEQ ID NO: 1 - protein, 132 aa, Staphylococcus aureus
HAKDASWLTSRKQLQPYGQYHGGGAHYGVDYAMPENSPVYSLTDGTVVQAGWSNYGGGNQVTIKEANSNNYQWYMHNNRLTVSAGDKVKAGDQIAYSGSTGNSTAPHVHFQRMSGGIGNQYAVDPTSYLQSR

SEQ ID NO: 2 - protein, 92 aa, Staphylococcus simulans
WKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 3 - protein, 17 aa, artificial
  LSS- mutated linker
AGYGKAASTVTPTPNTG

SEQ ID NO: 4 - protein, 17 aa, Staphylococcus simulans
AGYGKAGGTVTPTPNTG

SEQ ID NO: 5 - protein, 243 aa, artificial
  Chimeric AA sequence of Chimera A
MGHAKDASWLTSRKQLQPYGQYHGGGAHYGVDYAMPENSPVYSLTDGTVVQAGWSNYGGGNQVTIKEANSNNYQWYMHNNRLTVSAGDKVKAGDQIAYSGSTGNSTAPHVHFQRMSGGIGNQYAVDPTSYLQSRAGYGKAASTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 6 - DNA, 729 bp, artificial
  Chimera A coding sequence
atgggtcatgcgaaagacgcaagctggttaacaagtcgtaaacaactacaaccatatggacaatatcacggtggtggtgcgcattacggtgtcgactatgcaatgcctgaaaattcaccagtttactcattaactgatggtacagtagtacaagcaggttggagtaactatggtggcggcaatcaagtaacgattaaagaagcgaacagtaataactaccaatggtatatgcataataatcgtttaactgtttcagctggtgataaagtcaaagctggtgaccaaattgcatattcaggtagtacgggtaattcaacagcgcctcacgtacacttccaacgtatgtctggtggcatcggtaatcaatatgcagtagacccaacgtcatacttgcaaagtagagcaggatatggaaaagcagcttctacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaag

SEQ ID NO: 7 - protein, 243 aa, artificial
  AA sequence of Chimera B
MGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKAGYGKAASTVTPTPNTGHAKDASWLTSRKQLQPYGQYHGGGAHYGVDYAMPENSPVYSLTDGTVVQAGWSNYGGGNQVTIKEANSNNYQWYMHNNRLTVSAGDKVKAGDQIAYSGSTGNSTAPHVHFQRMSGGIGNQYAVDPTSYLQSR

SEQ ID NO: 8 - DNA, 729 bp, artificial
  Coding sequence of Chimera B
atgggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaaggcaggatatggaaaagcagcttctacagtaactccaacgccgaatacaggtcatgcgaaagacgcaagctggttaacaagtcgtaaacaactacaaccatatggacaatatcacggtggtggtgcgcattacggtgtcgactatgcaatgcctgaaaattcaccagtttactcattaactgatggtacagtagtacaagcaggttggagtaactatggtggcggcaatcaagtaacgattaaagaagcgaacagtaataactaccaatggtatatgcataataatcgtttaactgtttcagctggtgataaagtcaaagctggtgaccaaattgcatattcaggtagtacgggtaattcaacagcgcctcacgtacacttccaacgtatgtctggtggcatcggtaatcaatatgcagtagacccaacgtcatacttgcaaagtaga

SEQ ID NO: 9 - protein, 16 aa, artificial
  Penatrin
RQIKIWFQNRRMKWKK

SEQ ID NO: 10 - protein, 30 aa, artificial
  CCP2
WEAKLAKALAKALAKHLAKALAKALKACEA

SEQ ID NO: 11 - protein, 22 aa, artificial
  CPP 3
MVTVLFRRLRIRRASGPPRVRV

SEQ ID NO: 12 - protein, 18 aa, artificial
  CPP 4
KLALKLALKALKAALKLA

SEQ ID NO: 13 - protein, 12 aa, artificial
  CPP 5
RRQRRTSKLMKR

SEQ ID NO: 14 - protein, 18 aa, artificial
  CPP 6
LLIILRRRIRKQAHAHSK

SEQ ID NO: 15 - protein, 8 aa, artificial
  poly-arginine
RRRRRRRR

SEQ ID NO: 16 - protein, 13 aa, artificial
  HIV-TAT
GRKKRRQRRRPPQ

SEQ ID NO: 17 - protein, 27 aa, artificial
  CPP 9
GWTLNSAGYLLGKINLKALAALAKKIL

SEQ ID NO: 18 - protein, 21 aa, artificial
  CPP 10
AGYLLGKINLKALAALAKKIL

SEQ ID NO: 19 - protein, 9 aa, artificial
  CPP 11
RKKRRQRRR

(19) United States
(12) Patent Application Publication
(10) Pub. No.: US 2024/0409910 A1
(43) Pub. Date: Dec. 12, 2024

(54) RECOMBINANT POLYPEPTIDE FOR USE AS A MEDICINE, ANTISEPTIC AGENT, ANTIBACTERIAL AGENT, ANTI-INFLAMMATORY AGENT, COMPOSITIONS COMPRISING IT AND USES THEREOF

(71) Applicant: MIEDZYNARODOWY INSTYTUT BIOLOGII MOLEKULARNEJ I KOMORKOWEJ W WARSZAWIE, Warszawa (PL)

(72) Inventors: Izabela SABALA, Skierniewice (PL); Elzbieta JAGIELSKA, Warszawa (PL); Beata KUCZYNSKA, Warszawa (PL); Weronika AUGUSTYNIAK, Kosow Lacki (PL); Pawel MITKOWSKI, Legionowo (PL); Alicja WYSOCKA, Warszawa (PL)

(73) Assignee: MIEDZYNARODOWY INSTYTUT BIOLOGII MOLEKULARNEJ I KOMORKOWEJ W WARSZAWIE, Warszawa (PL)

(21) Appl. No.: 17/768,608

(22) PCT Filed: Oct. 13, 2020
(86) PCT No.: PCT/PL2020/050075

§ 371 Date: Apr. 13, 2022

(30) Foreign Application Priority Data
Oct. 13, 2019 (PL) P.431445

(51) Int. Cl. C12N 9/52; A01N 63/50; A01P 1/00; A61K 8/66; A61K 38/48; A61P 31/04; A61Q 17/00

(52) U.S. Cl. CPC C12N 9/52; A01N 63/50; A01P 1/00; A61K 8/66; A61K 38/4886; A61P 31/04; A61Q 17/005; C12Y 304/24075; C07K 2319/02

(57) ABSTRACT

The present invention relates to a recombinant polypeptide comprising a LytM catalytic domain, and a lysostaphin cell wall binding domain, optionally, an amino acid linker, and, optionally, a domain directing to the inside of an eukaryotic cell, for use as a medicine, antiseptic agent, antibacterial agent and/or anti-inflammatory agent. In particular, the invention relates to a recombinant polypeptide for use in the prevention and/or treatment of a dermatological disorder and/or inflammation. The recombinant polypeptide formulated for topical administration is also encompassed. A veterinary composition for intramammary administration comprising the recombinant polypeptide, in particular, for use in the treatment of mastitis, is also included. The invention also relates to the use of the recombinant polypeptide as a diagnostic reagent, disinfectant, antiseptic and antibacterial agent, as well as a pharmaceutical, cosmetic or care composition comprising thereof.

### FIELD OF THE INVENTION

The present invention relates to a recombinant polypeptide comprising a LytM catalytic domain, a cell wall binding domain from lysostaphin and, optionally, a peptide linker, and, optionally, a domain directing to the inside of an eukaryotic cell, for use as a medicine, antiseptic agent, antibacterial agent and/or anti-inflammatory agent. In particular, the invention relates to a recombinant polypeptide for use in the prevention and/or treatment of a dermatological disorder and/or inflammation. The invention also includes a veterinary composition for use as a medicine, antiseptic agent, antibacterial agent and/or anti-inflammatory agent, preferably formulated for intramammary administration, in particular, for use in the treatment of mastitis. The invention includes a pharmaceutical composition for use as a medicine, antiseptic, antimicrobial and/or anti-inflammatory agent, cosmetic or care composition containing a recombinant polypeptide for cosmetic, care and hygiene applications in humans and animals. The invention also relates to the use of the recombinant polypeptide as a diagnostic reagent, disinfectant, antibacterial agent, active agent in a cosmetic, care or hygienic composition, in humans and animals.

In particular, the invention relates to the use of the recombinant polypeptide in the prevention and/or treatment of dermatological disorders and/or inflammations. The recombinant polypeptide in a formulation for topical administration is also encompassed. This also includes a veterinary composition comprising the recombinant polypeptide. The veterinary composition according to the invention is intended for use in the prevention and/or treatment of a dermatological disorder and/or inflammation in a non-human animal. In particular, the invention relates to udder administration, particularly intramammary administration, and its use in the treatment of udder inflammation, particularly mastitis.

### STATE OF THE ART

Bacteria of the genus Staphylococcus (staphylococci) are responsible for a whole range of infections in humans and animals. Moreover, many Staphylococcus species are resistant to antibiotics; it is estimated that 30-50% of humans are carriers of these bacteria. Therefore, it is urgently required to provide alternative methods of treatment of infections caused by bacteria of the genus Staphylococcus. Even though antibiotics are still major therapeutic agents for the treatment of staphylococcal infections, they often lead to considerable side effects and further contribute to the emergence of antibiotic resistance. Therefore, new, alternative and effective methods of treatment of infections caused by staphylococci are urgently needed.

Dermatological and inflammatory disorders may be caused by various species of bacteria, including staphylococci. They can occur spontaneously or, e.g., in a wound, ulcer or burn. Infected wounds can be life-threatening. Additionally, biofilm producing bacteria can be present in the infected wounds, or wounds can be secondarily infected with bacteria (Bowler et al., 2001). Infections with antibiotic-resistant Staphylococcus aureus strains, such as MRSA (methicillin-resistant Staphylococcus aureus) strains are particularly dangerous. Staphylococcal infections are particularly common in the case of diabetic foot, pressure ulcers and burns.

Staphylococcus is also one of the causative agents of udder inflammation in dairy cows, known as mastitis. Mastitis is a persistent, inflammatory reaction of the mammary gland and udder tissue, and is a major endemic disease of dairy cattle, leading to annual losses of more than 1 billion Euro in Europe. Mastitis arises when leukocytes are released into the mammary gland, as an immune response to bacterial invasion of the teat canal by different bacteria. Milk-secreting tissues and ducts of the mammary glands are damaged by toxins released by bacteria. Published data show that more than half of the bacteria isolated from udders are Staphylococci (Malinowski and Smulski, 2007). S. aureus is responsible for both acute clinical and sub-clinical forms of mastitis, and particularly chronic infections. In cases of acute and chronic udder inflammation, especially caused by S. aureus, antibiotics are the standard-of-care treatment.

The infected cows are carriers of pathogenic bacteria, therefore several infected cows in a herd can form a persistent reservoir for future outbreaks of staphylococcal infections. In particular, S. aureus is rapidly transferred between the animals. Mastitis poses a serious threat to human health as well. Bacterial infections of milk and its products may cause food poisoning in humans who consume them.

One of the possible approaches to combat mastitis is the use of bacteriolytic enzymes. Although various bacteriolytic enzymes and chimeric versions thereof have been recently tested, none of them demonstrated a satisfactory enzymatic activity in raw cow's milk (see Table 1 below).

Chimeras composed of the LytM catalytic domain and the lysostaphin cell wall binding domain have been described earlier (Osipovitch and Griswold, 2015, Jagielska, Chojnacka et al., 2016), however, their activity in milk and serum was not demonstrated at that time.

This property is very rare and cannot be predicted based on the properties of individual domains that make up the chimera. Although many phage lysins and chimeric enzymes have been tested, even those that showed activity in physiological conditions and pasteurized milk, did not eliminate staphylococci from raw milk (Verbree, Datwyler et al., 2018, Rodriguez-Rubio, Martinez et al., 2013) and/or they lost their bacteriolytic properties in the presence of serum, which eliminates their practical application in veterinary medicine and medicine (Osipovitch, Therrien et al., 2015).

From the publication WO2012144912, a stable LytMCD derivative (LytM185-316) from Staphylococcus aureus is known, with the activity of glycylglycine endopeptidase against S. aureus bacteria under non-physiological conditions, at very low conductivity, below 10 mS/cm, low salt concentration and low temperature. This derivative of LytM185-316 lacks a bacterial cell wall binding domain and is also inactive under physiological conditions in in vivo staphylococcal infections such as mastitis or a staphylococcal wound.

From the publication WO2007/130655, a chimeric TAME polypeptide, which includes a cell wall lysing domain LytM and a lysostaphin cell wall binding domain is known, however, the chimera presented in sequence 9, in the light of the available information, cannot exhibit biological activity, as it does not include zinc coordinating amino acids in the active center, necessary for the activity of the LytM enzyme.

The chimeras presented in that publication do not show activity against Staphylococcus, in particular, Staphylococcus aureus under physiological conditions, which is whole milk, and are not able to act as a medicine, antibacterial agent and/or anti-inflammatory agent, which prevents or treats dermatological and/or inflammatory disorders, particularly those caused by bacteria of the genus Staphylococcus, in particular Staphylococcus aureus, particularly in an animal suffering from mastitis or at risk of developing such an inflammation.

### SUMMARY OF THE INVENTION

The recombinant polypeptide and the veterinary composition according to the present invention differ from the standard antibiotics in their specificity and activity against Staphylococcus strains. Unexpectedly, it was found that by combining the LytM185-316 catalytic domain with the lysostaphin cell wall binding domain (LssCWT), preferably via a peptide linker (Lss_linker), a recombinant polypeptide with strong antibacterial properties, especially against staphylococci, active under physiological conditions is obtained. The inventors of the present invention unexpectedly found that the recombinant polypeptide according to the invention is highly active in raw cow's milk, preferably in raw undiluted milk, and in serum, as well as in wounds infected by staphylococci. Moreover, an unexpectedly rapid and effective bacteriolytic activity against Staphylococcus strains was observed. This is a valuable property, considering the efficacy provided by the polypeptide according to the invention, and pharmaceutical compositions comprising thereof. Unlike most antibiotics, the recombinant polypeptide according to the invention does not require any metabolic activity of bacteria or bacterial growth to be effective, as it acts bacteriolytically upon contact with the bacterial cell wall. This property makes the compositions comprising the recombinant polypeptide, particularly the veterinary composition according to the present invention, suitable for quick reduction of bacterial count in the infected hosts. The inventors unexpectedly found that due to the above properties, the recombinant polypeptide and compositions comprising thereof, particularly the veterinary composition, can also solve the problem of antibiotic resistance in staphylococcal infections. Moreover, the inventors unexpectedly found that the recombinant polypeptide according to the invention is highly specific for Staphylococcus species and does not cause lysis of non-target bacteria, including commensal bacteria, thereby potentially reducing side effects.

In Table 1 below, a summary of the activities of the various peptidoglycan hydrolases is shown.

TABLE 1
Summary of the activity of peptidoglycan hydrolases comprising a cell wall binding domain from lysostaphin
(LssCWT) or without this domain, in milk and serum based on own research and literature data.

| Enzymes | | | | | | |
|---|---|---|---|---|---|---|
| | Binding | | | | | |
| Catalytic domain | domain | Activity | | | | |
| in the analyzed | from | PBS and | | serum | | |
| enzyme | LssCWT | UHT milk | raw milk | (%) | Comments | Literature |
| LytMCD | LssCWT | +++++ | +++++ | +++++ (100) | | This |
| | | | | | | application |
| | | | | | | (Chimera A |
| | | | | | | according to |
| | | | | | | the invention: |
| | | | | | | SEQ ID NO: 5) |
| Lss | LssCWT | +++++ | nt | +++ (60) | | Osipovitch, |
| | | | | | | Therrien et al., |
| | | | | | | 2015 |
| Isolated LytMCD | none | − | − | − | | WO012/144912 |
| domain | | | | | | A1 and own data |
| LytH | LssCWT | − | nt | nt | | Osipovitch, |
| LytO | LssCWT | +++ | nt | ++ (60) | | Griswold 2015 |
| SsaALP | LssCWT | ++ | nt | + (60) | | |
| LDP | LssCWT | − | nt | nt | | |
| PH | LssCWT | + | nt | nt | | |
| Lss | LssCWT | ++++ | ++* | nt | the best known to | Verbree et al., |
| CHAPK_{—} | LssCWT | ++++ | −* | nt | date among over 170 | 2018 |
| CHAPK_CHAPK_{—} | LssCWT | ++++ | nt | nt | peptidoglycan | |
| M23LST_CHAPH5 | LssCWT | ++++ | nt | nt | hydrolase chimeras | |
| CHAPH5 | LssCWT | ++ | nt | nt | tested | |

− none;
+ <20%,
++ 21-40%,
+++ 41-60%,
++++ 61-80%,
+++++ 81-100% of lysostaphin activity under optimal conditions,
*test carried out with 10³ bacterial cells

The invention relates to recombinant polypeptides and compositions comprising thereof, for use as a diagnostic reagent, disinfectant, antiseptic agent, medicine, antibacterial agent and/or anti-inflammatory agent, which prevents or treats dermatological and/or inflammatory disorders, antibacterial agent, as well as an active agent in cosmetic, care or hygienic compositions for humans and animals. In particular, the inventors have found that the recombinant polypeptides are effective in treating mastitis in ruminants, in particular, dairy cows. Experimental data provided below prove the recombinant polypeptide to be effective in treating mastitis in ruminants by effectively binding to the outer surface of bacteria of the genus Staphylococcus, in particular Staphylococcus aureus, causing cell lysis and thereby eliminating the bacteria.

The inventors have further shown in vivo that the recombinant polypeptides and compositions comprising them are effective in combating infections with bacteria of the genus Staphylococcus, in particular Staphylococcus aureus, particularly skin infections and infected wounds, particularly in the topical treatment of dermatological diseases, wounds and conditions of skin and/or soft tissues.

One aspect of the invention relates to a recombinant polypeptide comprising (i) a LytM catalytic domain (LytMCD), comprising an amino acid sequence shown in SEQ ID NO: 1 or having at least 80% identity thereto, (ii) a lysostaphin cell wall binding domain, binding the cell wall of bacteria (LssCWT), comprising an amino acid sequence shown in SEQ ID NO: 2 or having at least 80% identity thereto, and (iii) optionally, a peptide linker, linking the LytM catalytic domain and the lysostaphin cell wall binding domain, for use as a medicine, antiseptic agent, antibacterial agent and/or anti-inflammatory agent.

In the preferred recombinant polypeptide for the abovementioned use, the LytM catalytic domain consists of the amino acid sequence shown in SEQ ID NO: 1.

In the preferred recombinant polypeptide for the abovementioned use, the lysostaphin cell wall binding domain consists of the amino acid sequence shown in SEQ ID NO: 2.

In the preferred recombinant polypeptide for the abovementioned use, the peptide linker, linking the LytM catalytic domain and the lysostaphin cell wall binding domain, comprises the amino acid sequence shown in SEQ ID NO: 3 or SEQ ID NO: 4, preferably, it consists of the sequence SEQ ID NO: 3.

The recombinant polypeptide for the abovementioned use, preferably comprises the sequence SEQ ID NO: 5 or is encoded by a nucleotide sequence comprising SEQ ID NO: 6, preferably, it consists of SEQ ID NO: 5.

The recombinant polypeptide for the abovementioned use, preferably comprises the sequence SEQ ID NO: 7 or is encoded by a nucleotide sequence comprising SEQ ID NO: 8, preferably, it consists of SEQ ID NO: 7.

The recombinant polypeptide for the abovementioned use, preferably further comprises, from the LssCWT domain side, at the N or C terminus of the polypeptide, an attached CCP signal sequence directing it to the inside of an eukaryotic cell, preferably the signal sequence is selected from SEQ ID NO: 9-19.

The recombinant polypeptide for the abovementioned use, is preferably used in the prevention and/or treatment of dermatological disorders and/or inflammation.

The recombinant polypeptide for the abovementioned use, is preferably used in the prevention and/or treatment of a dermatological disorder and/or inflammation, which involves the presence of Gram-positive bacteria, preferably bacteria of the genus Staphylococcus, most preferably Staphylococcus aureus.

The recombinant polypeptide for the abovementioned use, is preferably used in a dermatological disorder and/or inflammation, which are selected from the group consisting of acne, pimples, dermatitis, preferably atopic dermatitis, infections and/or injuries of a mucous membrane or the skin, wounds, diabetic foot, ulcers, burns and an inflammation of the mammary gland, udder, eye, ear, throat, sinuses or genitals.

The recombinant polypeptide for the abovementioned use, preferably exhibits bacteriolytic activity in raw milk and/or serum, wherein the raw milk is preferably raw cow's milk, and in which the serum is preferably bovine or human serum.

The recombinant polypeptide for the abovementioned use, is preferably formulated for topical administration, preferably in the form of a cream, lotion, solution, ointment, gel, foam, transdermal patch, powder, paste, suspension, tape, pad, swab, wound dressing or compress.

The recombinant polypeptide for the abovementioned use, is preferably formulated for intramammary (including intra-nipple/teat/udder) delivery.

The recombinant polypeptide for the abovementioned use, is preferably delivered to a nipple or teat, to udder, by infusion, rinsing or dipping.

The recombinant polypeptide for the abovementioned use, is preferably delivered to a female human or non-human animal suffering from mastitis or at risk of developing mastitis, preferably a ruminant, more preferably a dairy cow.

Typically, the recombinant polypeptide is used for the prevention and/or treatment of a dermatological disorder and/or inflammation.

In some embodiments, the dermatological and/or inflammatory disorders involve the presence of a Gram-positive bacterium, preferably a bacterium of the genus Staphylococcus, most preferably Staphylococcus aureus.

In some embodiments, the recombinant polypeptide is used in the prevention and/or treatment of a dermatological and/or inflammatory disorder, wherein the dermatological disorder is selected from the group consisting of acne, pimples, dermatitis, preferably atopic dermatitis, infections and/or injuries of a mucous membrane, skin, wounds, diabetic foot, ulcers, burns and an inflammation of the mammary gland, udder, eye, ear, throat, sinuses or genitals.

Preferably, the recombinant polypeptide exhibits bacteriolytic activity in raw milk and/or serum, wherein the raw milk is preferably raw cow's milk, and wherein the serum is preferably bovine or human serum. Thus, the recombinant polypeptide according to the invention, is preferably contacted with the bacteria to be eliminated from milk, most preferably raw milk, or from serum or blood or from eukaryotic cells.

In some embodiments, the recombinant polypeptide according to the present invention may be formulated for topical administration. Preferably, the formulation is in the form of a cream, lotion, solution, ointment, gel, foam, transdermal patch, powder, paste, tincture, tape, pad, swab, wound dressing or compress.

In some embodiments, the recombinant polypeptide is formulated for intramammary delivery.

In some embodiments, the recombinant polypeptide is delivered to a nipple or teat, preferably intramammary (including), preferably by infusion, rinsing or dipping.

The preferred embodiments relate to the delivery of the recombinant polypeptide to a female human or non-human animal suffering from mastitis or at risk of developing mastitis, preferably a ruminant, more preferably a dairy cow.

Another aspect of the invention relates to a veterinary composition comprising a recombinant polypeptide, comprising (i) a LytM catalytic domain (LytMCD), comprising an amino acid sequence shown in SEQ ID NO: 1 or having at least 80% identity thereto, (ii) a lysostaphin domain, binding the cell wall of bacteria (LssCWT), comprising an amino acid sequence shown in SEQ ID NO: 2 or having at least 80% identity thereto, and (iii) optionally, a peptide linker, linking the LytM catalytic domain and the lysostaphin cell wall binding domain, at least one excipient approved for use in pharmaceutical products, for use as a medicine, antiseptic agent, antibacterial agent and/or anti-inflammatory agent.

In the preferred veterinary composition for the abovementioned use, in a recombinant polypeptide, the LytM catalytic domain consists of the amino acid sequence shown in SEQ ID NO: 1.

In the preferred veterinary composition for the abovementioned use, in a recombinant polypeptide, the lysostaphin cell wall binding domain consists of the amino acid sequence shown in SEQ ID NO: 2.

In the preferred veterinary composition for the abovementioned use, in a recombinant polypeptide, the peptide linker, linking the LytM catalytic domain and the lysostaphin cell wall binding domain, comprises the amino acid sequence shown in SEQ ID NO: 3 or SEQ ID NO: 4, preferably, it consists of the sequence SEQ ID NO: 3.

In the preferred veterinary composition for the abovementioned use, a recombinant polypeptide comprises the sequence SEQ ID NO: 5 or is encoded by a nucleotide sequence comprising SEQ ID NO: 6, preferably, it consists of SEQ ID NO: 5.

In the preferred veterinary composition for the abovementioned use, a recombinant polypeptide comprises the sequence SEQ ID NO: 7 or is encoded by a nucleotide sequence comprising SEQ ID NO: 8, preferably, it consists of SEQ ID NO: 7.

In the preferred veterinary composition for the abovementioned use, a recombinant polypeptide, from the LssCWT domain side, at the N or C terminus of the polypeptide, an attached CCP signal sequence directing to the inside of an eukaryotic cell, preferably the signal sequence is selected from SEQ ID NO: 9-19.

The veterinary composition for the abovementioned use, is intended, preferably, for use in the prevention and/or treatment of a dermatological disorder and/or inflammation.

The veterinary composition for the abovementioned use, is preferably used in a dermatological disorder and/or inflammation, which is selected from the group consisting of dermatitis, injuries and/or infections of a mucous membrane or of the skin, wounds, ulcers, and inflammation of the mammary gland, inflammation of an eye, ear, throat, sinuses or genitals.

The veterinary composition for the abovementioned use, is preferably formulated for intramammary delivery.

The veterinary composition for the abovementioned use, is preferably used in the treatment of mastitis in ruminants.

Specific embodiments relate to the veterinary composition for use in the treatment of mastitis in ruminants.

Specific embodiments relate to the veterinary composition for use in the prevention and/or treatment of a dermatological disorder and/or inflammation.

The preferred embodiments relate to the veterinary composition, wherein a dermatological disorder and/or inflammation is selected from the group consisting of dermatitis, injuries and/or infections of a mucous membrane or of the skin, wounds and inflammation of the udder, eye, ear, throat, sinuses or genitals.

In another embodiment, the veterinary composition is intended for intramuscular administration.

In yet another embodiment, the veterinary composition is intended for use in the treatment of mastitis in ruminants.

Preferably, the veterinary or other pharmaceutical composition comprising the recombinant polypeptide according to the invention is contacted with the bacteria to be eliminated from milk, most preferably from raw milk, or from serum or blood.

The invention also relates to the use of a recombinant polypeptide comprising (i) a LytM catalytic domain (LytMCD), comprising an amino acid sequence shown in SEQ ID NO: 1 or having at least 80% identity thereto, (ii) a lysostaphin domain, binding the cell wall of bacteria (LssCWT), comprising an amino acid sequence shown in SEQ ID NO: 2 or having at least 80% identity thereto, and (iii) optionally, a peptide linker, linking the LytM catalytic domain and the lysostaphin cell wall binding domain, as a diagnostic reagent, disinfectant, antibacterial agent.

In the preferred use of the recombinant polypeptide, the LytM catalytic domain consists of the amino acid sequence shown in SEQ ID NO: 1.

In the preferred use of the recombinant polypeptide, the lysostaphin cell wall binding domain consists of the amino acid sequence shown in SEQ ID NO: 2.

In the preferred use of the recombinant polypeptide, the peptide linker, linking the LytM catalytic domain and the lysostaphin cell wall binding domain, comprises the amino acid sequence shown in SEQ ID NO: 3 or SEQ ID NO: 4, preferably, it consists of the sequence SEQ ID NO: 3.

In the preferred use of the recombinant polypeptide, it comprises the sequence SEQ ID NO: 5 or is encoded by a nucleotide sequence comprising SEQ ID NO: 6, preferably, it consists of SEQ ID NO: 5.

In the preferred use of the recombinant polypeptide, it comprises the sequence SEQ ID NO: 7 or is encoded by a nucleotide sequence comprising SEQ ID NO: 8, preferably, it consists of SEQ ID NO: 7.

In the preferred use of the recombinant polypeptide, it further comprises, from the LssCWT domain side, at the N or C terminus of the polypeptide, an attached CCP signal sequence directing to the inside of an eukaryotic cell, preferably the signal sequence is selected from SEQ ID NO: 9-19.

In the preferred use of the recombinant polypeptide, when it is used as a diagnostic reagent, it is used in Staphylococcus diagnostics, preferably Staphylococcus aureus.

In the preferred use of the recombinant polypeptide, when is used as a disinfectant, it is used for disinfecting surfaces, appliances and equipment, furniture, floors, particularly in hospitals, medical and dental offices, kitchens and bathrooms, particularly for disinfecting surfaces from Staphylococcus, preferably Staphylococcus aureus.

In the preferred use of the recombinant polypeptide, when the polypeptide is used as an antibacterial agent, it is used against Staphylococcus, preferably Staphylococcus aureus.

In the preferred use of the recombinant polypeptide, it is used as an antibacterial agent in milk, serum, preferably in whole/raw milk.

In the preferred use of the recombinant polypeptide, when the polypeptide is used as an active agent in a cosmetic, care or hygienic composition for humans or animals, it is used as an active agent against Staphylococcus, preferably Staphylococcus aureus.

The invention also relates to a cosmetic or care composition for cosmetic, care and hygienic applications in humans or animals, which comprises a recombinant polypeptide comprising (i) a LytM catalytic domain (LytMCD), comprising an amino acid sequence shown in SEQ ID NO: 1 or having at least 80% identity thereto, (ii) a lysostaphin domain, binding the cell wall of bacteria (LssCWT), comprising an amino acid sequence shown in SEQ ID NO: 2 or having at least 80% identity thereto, and (iii) optionally, a peptide linker, linking the LytM catalytic domain and the lysostaphin cell wall binding domain, wherein the composition additionally comprises at least one carrier approved for care and hygienic applications in humans or animals, and is intended for external use.

Preferred is a cosmetic or care composition, in which, in the recombinant polypeptide, the LytM catalytic domain consists of the amino acid sequence shown in SEQ ID NO: 1, equally preferred, in which, in the recombinant polypeptide, the lysostaphin cell wall binding domain consists of the amino acid sequence shown in SEQ ID NO: 2, equally preferred is a composition, in which, in the recombinant polypeptide, the peptide linker, linking the LytM catalytic domain and the lysostaphin cell wall binding domain comprises the amino acid sequence shown in SEQ ID NO: 3 or SEQ ID NO: 4, preferably, it consists of the sequence SEQ ID NO: 3, equally preferred is a composition, in which the recombinant polypeptide comprises the sequence SEQ ID NO: 5 or is encoded by a nucleotide sequence comprising SEQ ID NO: 6, preferably, it consists of SEQ ID NO: 5, equally preferred is a composition, in which the recombinant polypeptide comprises the sequence SEQ ID NO: 7 or is encoded by a nucleotide sequence comprising SEQ ID NO: 8, preferably, it consists of SEQ ID NO: 7.

The preferred cosmetic or care composition is used to limit the occurrence or to remove bacteria of the genus Staphylococcus, most preferably Staphylococcus aureus.

The invention also relates to a pharmaceutical composition comprising a recombinant polypeptide comprising (i) a LytM catalytic domain (LytMCD), having the amino acid sequence shown in SEQ ID NO: 1 or having at least 80% identity thereto, (ii) a lysostaphin cell wall binding domain of (LssCWT), comprising or having at least 80% identity thereto, the amino acid sequence shown in SEQ ID NO: 2, and (iii) optionally a peptide linker, linking the LytM catalytic domain and the bacterial wall binding domain of lysostaphin, and a pharmaceutically acceptable excipient for use as a medicine, an antiseptic, antibacterial and/or anti-inflammatory agent.

In the preferred pharmaceutical composition for use as a medicine, antiseptic, antibacterial and/or anti-inflammatory agent in the recombinant peptide, the LytM catalytic domain consists of the amino acid sequence shown in SEQ ID NO: 1.

In the preferred pharmaceutical composition for use as a medicine, antiseptic, antibacterial and/or anti-inflammatory agent in a recombinant peptide, the lysostaphin cell wall binding domain consists of the amino acid sequence shown in SEQ ID NO: 2.

In the preferred pharmaceutical composition for use as a medicine, antiseptic, antibacterial and/or anti-inflammatory agent in the recombinant peptide, the peptide linker, linking the LytM catalytic domain and the lysostaphin domain comprises the amino acid sequence shown in SEQ ID NO: 3 or SEQ ID NO: 4, preferably, it consists of the sequence SEQ ID NO: 3.

In the preferred pharmaceutical composition for use as a medicine, antiseptic, antibacterial and/or anti-inflammatory agent, the recombinant peptide comprises the sequence of SEQ ID NO: 5 or is encoded by a nucleotide sequence comprising SEQ ID NO: 6, preferably, it consists of SEQ ID NO: 5.

In the preferred pharmaceutical composition for use as a medicine, antiseptic, antibacterial and/or anti-inflammatory agent, the recombinant peptide comprises the sequence of SEQ ID NO: 7 or is encoded by a nucleotide sequence comprising SEQ ID NO: 8, preferably consists of SEQ ID NO: 7.

In the preferred pharmaceutical composition for use as a medicine, antiseptic, antibacterial and/or anti-inflammatory agent, a recombinant polypeptide, from the LssCWT domain side, at the N or C terminus of the polypeptide, an attached CCP signal sequence directing to the inside of an eukaryotic cell, preferably the signal sequence is selected from SEQ ID NO: 9-19.

The pharmaceutical composition is preferably used to prevent and/or treat a disease and/or inflammation caused by bacteria of the Staphylococcus genus, most preferably Staphylococcus aureus.

The pharmaceutical composition for use as a medicine, antiseptic, antibacterial and/or anti-inflammatory agent is preferably in the form of a cream, suspension, lotion, solution, ointment, gel, foam, transdermal patch, powder, paste, suspension, tape, pad, swab, wound dressing or compress. Publications cited in the description, and the references given therein, are also incorporated herein as references.

### DESCRIPTION OF DRAWINGS

For a better understanding of the invention, it has been illustrated in the embodiments and in the accompanying figures, in which:

FIG. 1: A) Schematic representation of domain organization in Chimeras A and B. Numbers in boxes represent amino acid positions in full-length proteins according to UniProt (https://www.uniprot.org/). LytMCD stands for the LytM catalytic domain (UniProt 033599), and LssCWT stands for the lysostaphin bacterial cell wall binding/targeting domain (Lss; UniProt P10547); B) shows the amino acid and nucleotide sequence of Chimera A, respectively; C) shows the amino acid and nucleotide sequence of Chimera B, respectively. Designations in B and C, respectively, underlined-sequences of the vector, light gray background—the CD domain of LytM, dark gray background—the CWT domain of Lss, unchecked-linker; D) schematic structure of Chimera A connected to the signal sequence at the C-terminus: Chimera A (LytM CD+LSS linker+LSS CWT)+C-terminal CPP and exemplary sequences of such chimeras; E) schematic structure of Chimera B connected to the signal sequence at the N-terminus: N-terminal CPP+Chimera B (LSS CWT+LSS linker+LytM CD), the signal sequences attached at the N-terminus can be, for example, the sequences shown in Table 1.

FIG. 2: Comparison of the lytic activity of Chimeras A and B demonstrates comparable efficacy of these two enzymes.

FIG. 3: Chimera A activity in UHT (ultra-heat temperature processing, ultra-heat treatment, or ultra-pasteurization) milk at 37° C. Time-kill assay in milk spiked with 10³ CFU/ml of S. aureus cells (strain NCTC 8325-4) in the presence of 36 nM, 53 nM, 100 nM, 360 nM and 4000 nM of Chimera A Samples were incubated at 37° C. for 24 h. Negative control was carried out without the addition of the enzyme. The experiment was performed twice (mean±SD).

FIG. 4: 10³ CFU/ml of S. aureus (strain NCTC 8325-4) were inoculated into a UHT whole milk (A) and incubated for 1 hour at 37° C. Chimera A caused complete elimination of bacteria in UHT whole milk (B).

FIG. 5: Chimera A activity in UHT whole milk at 4° C. (A) and 22° C. (B). S. aureus cells (strain NCTC 8325-4; 10⁶ CFU/ml) were incubated with 100 nM of Chimera A for 24 hours. The negative control did not include the chimeric enzyme. The experiment was performed three times.

FIG. 6: Chimera A activity in raw bovine milk (undiluted) at 37° C. A) Time-kill assay in milk with the addition of 10³ CFU/ml of S. aureus cells (strain NCTC 8325-4) in the presence of either 36 nM or 100 nM of Chimera A. Samples were incubated at 37° C. for 3 hours; B) time-kill assay in milk with the addition of 10⁶ CFU/ml of S. aureus cells (strain NCTC 8325-4) and 100 nM of Chimera A. Samples were incubated at 22° C. for 24 hours, taking samples to assess the number of bacteria at time 0, after two hours and at the end of the experiment. In both the experiments, the negative control did not contain Chimera A. The experiments were performed twice (mean±SD).

FIG. 7: 10³ CFU/ml of S. aureus cells (strain NCTC 8325-4) were inoculated into raw, undiluted cow's milk and incubated for 1 hour at 37° C. with or without the addition of Chimera A. Assessment of staphylococcal cell number in the inoculated milk samples before the enzyme treatment (A), after 1 hour of incubation with Chimera A (B), after 1 hour of incubation without Chimera A (C).

FIG. 8: Somatic cell count measured before and after 7 days of treatment with Chimera A.

FIG. 9: Bacteriolytic activity of Chimera A in the presence of fetal bovine serum (FBS). The activity of 100 nM of Chimery A was estimated as a reduction of turbidity of bacterial cells suspension within 1 hour at room temperature.

FIG. 10. Alignment of the amino acid sequence of Chimera A and the chimera known in the state of the art. Zinc-coordinating amino acids in the active center, necessary for integrity and activity of the enzyme, are highlighted in gray.

FIG. 11. Comparison of the lytic activity of Chimera B, with and without the CPP sequence, against S. aureus under different environmental conditions: A—glycine buffer, B—bovine serum, C—UHT milk.

FIG. 12. Antimicrobial effect of a hydrogel wound dressing, with or without a Chimera according to the invention, on the treatment of wounds infected by staphylococci. The graph shows the number of bacterial cells counted. Each dot represents one mouse; horizontal bar, calculated mean value of S. aureus in the isolates from infected wounds. Black circle—negative control, and white circle with a black outline tested sample (results shown for Chimera A).

FIG. 13. In vivo antimicrobial and anti-inflammatory effects of a hydrogel wound dressing with a recombinant polypeptide according to the invention (Chimera A).

### DETAILED DESCRIPTION OF THE INVENTION

The following explanations and general definitions relate to the descriptions of preferred embodiments of the invention, given below.

The present invention as illustratively described in the following may suitably be practiced in the absence of any element or elements, limitation or limitations, not specifically disclosed herein.

The present invention will be described with respect to particular embodiments and with reference to certain figures, but the invention is not limited thereto, and is limited only by the claims.

When the term “comprising” or “including” is used in this description and claims, it does not exclude other elements. For the purposes of the present invention, the term “consisting of” is considered a preferred embodiment of the term “comprising”, “including”. If a group is defined below, which contains at least a certain number of embodiments, this should also be understood as disclosing a group, which preferably consists of only these embodiments.

The terms “about” or “approximately” in the context of the present invention stand for a range of accuracy that a person skilled in the art will understand to be sufficient to still provide the technical effect of the given feature. The term “typically” is to be understood as a deviation from the indicated numerical value of ±10%, preferably ±5%.

Technical terms are used by their typical meaning. If a specific meaning is conveyed to certain terms, the meaning will be described in detail in reference to the context in which the given term was used.

One aspect of the invention relates to a recombinant polypeptide comprising (i) a LytM catalytic domain, comprising an amino acid sequence shown in SEQ ID NO: 1 or having at least 80% identity thereto, (ii) a lysostaphin cell wall binding domain, comprising an amino acid sequence shown in SEQ ID NO: 2 or having at least 80% identity thereto, and (iii) optionally, a peptide linker, linking the LytM catalytic domain and the lysostaphin cell wall binding domain for use as a diagnostic reagent, disinfectant, antiseptic agent, antibacterial agent and/or anti-inflammatory agent.

In one embodiment, the recombinant polypeptide comprises (i) a LytM catalytic domain, comprising an amino acid sequence shown in SEQ ID NO: 1 or having at least 80%, 85%, 90%, 92%, 94%, 95%, 96%, 97%, 98% or 99% sequence identity thereto, (ii) a lysostaphin cell wall binding domain, comprising an amino acid sequence shown in SEQ ID NO: 2 or having at least 80%, 85%, 90%, 92%, 94%, 95% 96%, 97%, 98% or 99% sequence identity thereto, and (iii) optionally, a peptide linker, linking the LytM catalytic domain and the lysostaphin cell wall binding domain, for use as a diagnostic reagent, disinfectant, antiseptic agent, antibacterial agent and/or anti-inflammatory agent.

The term “recombinant” used herein, refers to a protein that is produced in a host cell which is genetically engineered to express the protein. The host can be of viral, mammalian, insect, yeast, bacterial, algal origin. Typically, the host cells are grown in a cell culture by standard procedures and the protein is subsequently obtained from the cell culture by harvesting and, optionally, further purified, by standard techniques such as ion exchange chromatography, affinity chromatography or molecular filtration.

In the present disclosure, the host cell is preferably an E. coli cell or a Pichia pastoris cell, most preferably an E. coli cell.

The term “polypeptide”, “peptide” or “protein” refers to a polymer in which most or all of the monomers are amino acids and are joined together by peptide bonds. When the amino acids are α-amino acids, either the L-optical isomer or the D-optical isomer can be used.

Additionally, amino acids not found in nature, e.g., β-alanine, phenylglycine and homoarginine are also included. The amino acids used in the present invention may be the D- or L-isomers, or mixtures thereof.

The term “recombinant polypeptide” according to the invention refers to the chimeric protein containing (i) a LytM catalytic domain, comprising an amino acid sequence shown in SEQ ID NO: 1 or having at least 80% identity thereto, (ii) a lysostaphin cell wall binding domain, comprising an amino acid sequence shown in SEQ ID NO: 2 or having at least 80% identity thereto, and (iii) optionally, a peptide linker, linking the LytM catalytic domain and the lysostaphin cell wall binding domain. The recombinant polypeptides of the present invention exhibit bacteriolytic activity against bacteria of the genus Staphylococcus, including S. aureus.

A LytM catalytic domain refers to a region of the enzyme that interacts with its substrate to cause the enzymatic reaction. LytM is a peptidoglycan hydrolase (autolysin) with glycyl-glycine endopeptidase activity. It acts specifically on polyglycine interpeptide bridges of staphylococcal cell wall peptidoglycan.

Typically, the LytM catalytic domain is defined by the amino acid sequence shown in SEQ ID NO: 1, or having at least 80%, 85%, 90%, 92%, 94%, 95%, 96%, 97%, 98%, or 99% sequence identity thereto.

Preferably, the catalytic domain originates from the LytM enzyme from S. aureus.

In the recombinant polypeptides according to the invention, the LytM catalytic domain is fused with the lysostaphin cell wall binding domain, optionally via a peptide linker, as disclosed herein. Thereby, the catalytic domain is capable of cleaving the cell wall with the aid of the cell wall binding domain targeting the chimeric enzyme to the cell wall of bacteria, preferably Staphylococcus, even more preferably Staphylococcus aureus. In a particularly preferred embodiment, the pentaglycine cross bridges of Staphylococcus aureus peptidoglycan are cleaved by the chimeric enzyme comprising the LytM catalytic domain and the lysostaphin cell wall binding domain.

A lysostaphin cell wall binding domain refers to a motif, which recognizes and binds with the bacterial outer surface. Lysostaphin (EC 3.4.24.75; glycyl-glycine endopeptidase) is a metalloendopeptidase from S. simulans.

In Gram-positive bacteria, the outer surface of the bacteria is typically the murein/peptidoglycan layer. Thus, the preferred binding element for these targets will be cell surface entities, whether peptide, protein, lipid, sugar, or a combination thereof. Preferably, the cell walls of Staphylococcus are cleaved, particularly preferably the cell walls of Staphylococcus aureus are cleaved. Typically, the lysostaphin cell wall binding domain is defined by the amino acid sequence shown in SEQ ID NO: 2, or having at least 80%, 85%, 90%, 92%, 94%, 95%, 96%, 97%, 98%, or 99% sequence identity thereto.

The cell wall binding domain is fused with the LytM catalytic domain, optionally via a peptide linker, as disclosed herein. Thus, the cell wall binding domain serves to direct the catalytic domain to the cell wall of the bacterium to be eliminated, preferably Staphylococcus, particularly preferably Staphylococcus aureus.

Optionally, a peptide linker links the LytM catalytic domain and the lysostaphin cell wall binding domain. Optionally means that the present invention works also in the absence of the peptide linker, for example, by direct fusion of the catalytic domain and cell wall binding domain. Typically, the peptide linker comprises from 5 to 35 amino acids, preferably from 10 to 30 amino acids, even more preferably, from 15 to 25 amino acids. In a preferred embodiment, the linker comprises about 17 amino acids.

The peptide linker used in the chimeric polypeptide according to the invention may have any amino acid sequence because it is involved neither in the cell wall targeting function of the cell wall binding domain nor in the enzymatic function of the catalytic domain of the chimeric polypeptide, but serves as a linker that most likely provides steric flexibility of the two domains within the polypeptide.

Preferably, the linker comprises the amino acid sequence AGYGKAASTVTPTPNTG (SEQ ID NO: 3) or a sequence having at least 70%, 75%, 80%, 85%, 90%, 92%, 94%, 95%, 96%, 97%, 98%, or 99% sequence identity thereto.

The wild-type linker sequence from lysostaphin (UniProt P10547) comprising the amino acid sequence AGYGKAGGTVTPTPNTG (SEQ ID NO: 4) can also be used as the linker.

The term “diagnostic reagent” refers to a diagnostic reagent detecting staphylococcal presence or absence in a sample, wherein binding of the recombinant polypeptide according to the invention to bacteria of the genus Staphylococcus is detected via a signal generating moiety attached to the recombinant polypeptide. The signal may also result from lysis of the staphylococcal cells by the recombinant polypeptide according to the invention. This might be detected either by changes in optical density of bacterial cultures, release of nucleic acids detected by colorimetric or fluorimetric markers, changes in conductivity, release of particular substances that can be detected in specific assays or any other biochemical, biophysical or optical method.

The diagnostic reagent may be part of a diagnostic kit.

The sample may be blood, saliva, urine, water, food (milk, milk products, meat, fish, sea food etc.), swaps taken from various parts of human or animal body, as well as any surfaces which might be contaminated/colonized by bacteria of the genus Staphylococcus.

The term “disinfectant” refers to a composition that destroys vegetative forms of microorganisms, such as bacteria, especially on inanimate objects. The disinfectant according to the present invention comprises the recombinant polypeptides as defined above. Thus, the disinfectant of the present invention acts by binding to the outer surface of bacteria of the genus Staphylococcus, ultimately lysing, and therefore eliminating them. Preferably, the bacteria are Staphylococcus aureus. Typically, disinfectants are used on inanimate objects, such as devices and equipment, surfaces of any kind, e.g., of furniture, floors. They are commonly used in hospitals, reception rooms and dental offices, sports facilities, production lines, in food factories and processing plants, as well as in kitchens and bathrooms.

The term “antiseptic agent” refers to a composition that prevents or inhibits the growth of microorganisms, such as bacteria. The antiseptic according to the present invention comprises the recombinant polypeptides as defined above. Thus, the antiseptic agent according to the present invention acts by binding to the outer surface of bacteria of the genus Staphylococcus, ultimately lysing, and therefore eliminating them. Preferably, the bacteria are Staphylococcus aureus. Typically, antiseptic agents are used on living tissue or skin to reduce the possibility of infection or sepsis.

The term “antibacterial agent” refers to an agent, which is directed against the outer surface of bacteria and therefore, lysing and effectively killing bacteria. The antibacterial agent comprises pharmaceutical or veterinary compositions. The antibacterial agent according to the present invention comprises the recombinant polypeptides as defined above. The antibacterial agent may simultaneously act as an anti-inflammatory agent.

The term “anti-inflammatory agent” refers to an agent counteracting inflammation. The anti-inflammatory agent according to the present invention counteracts inflammation by targeting the cell wall of bacteria and killing the bacteria, thus not eliciting an immune response, and therefore, not provoking inflammation. The anti-inflammatory agent according to the present invention comprises the recombinant polypeptides as defined above. The anti-inflammatory agent may simultaneously act as an antibacterial agent.

Typically, the recombinant polypeptide according to the invention is used for the prevention and/or treatment of a disorder caused by bacteria of the genus Staphylococcus, particularly a dermatological disorder and/or inflammation.

The term “prevention” refers to a reduction in risk of acquiring or developing a disorder caused by bacteria of the genus Staphylococcus, particularly a dermatological disorder and/or inflammation, i.e. causing at least one of the clinical symptoms not to occur in a human or non-human animal that may be exposed to bacteria of the genus Staphylococcus. Prevention also relates to a reduction in risk of acquiring or developing a disorder caused by bacteria of the genus Staphylococcus, particularly a dermatological disorder and/or inflammation, i.e. causing any clinical symptoms not to occur in a human or non-human animal that carries bacteria of the genus Staphylococcus. Hence, prevention also refers to the elimination of bacteria of the genus Staphylococcus in human or non-human animal carriers that do not experience any clinical symptoms. In other words, limiting or preventing the transmission of bacteria of the genus Staphylococcus from one carrier to another is also encompassed by the term “prevention”.

The term “treatment” refers to the treatment of a disorder caused by bacteria of the genus Staphylococcus, or in which these bacteria are involved, particularly a dermatological disorder and/or inflammation, in particular, in one embodiment, the treatment relates to ameliorating the disease and/or condition (i.e., arresting the development of the disease or reducing the extent or severity of at least one of the clinical symptoms thereof). Treatment can also refer to ameliorating at least one physical parameter, which may not be discernible by the human or non-human animal. Treatment may also refer to changes in the symptoms of the disorder: physical, (e.g., stabilization of a discernible symptoms), physiological, (e.g., stabilization of a physical parameter), or both. Alternatively, “treatment” can also refer to slowing the progression of the disorder.

Dermatological disorders are disorders which affect the skin, which is made of layers of ectodermal tissue. The skin is in contact with the environment, and it protects the underlying structures (e.g., muscles, bones, ligaments and internal organs) from contact with the outside environment or from injury, including protection against pathogens such as bacteria. The skin may be affected by disorders that are diverse in terms of cause, presentation and/or treatment. Preferably, in the embodiments, the dermatological disorders are acne, pimples, dermatitis, atopic dermatitis, infections and/or injuries of a mucous membrane or of the skin, wounds, diabetic foot, ulcers, burns and inflammation of the mammary gland, udder, eyes, ears, throat, sinuses or genitals. The mucous membrane is to be understood as the thin skin that produces mucus and covers the inside surface of a part of the body. The mucous membrane comprises one or more epithelial layers such as within the teat/nipple, lactiferous ducts, lactiferous sinus, eye, ear, nasal or respiratory passage and genitals. Diabetic foot is a disease linked with type II diabetes and is associated with a high risk for skin lesions and wounds on the skin of the feet which often develop into chronic wounds. Ulcers are a discontinuity of the skin or a break in the skin or mucous membranes. Ulcers comprise diabetic foot ulcers, a complication of diabetic foot, pressure ulcers, genital ulcers, ulcerative dermatitis and anal fissures. Inflammation is a causative agent of ulcers. The dermatological disorder can occur in isolation or in combination with inflammation as described below. Hence, the dermatological disorder may be associated with inflammation.

Inflammations are disorders which affect the organs by inflammation, i.e. a local response to cellular injury that is manifested by any one or a combination of the following: capillary dilatation, leukocytic infiltration, redness, heat, and pain. Inflammation serves as a mechanism initiating the elimination of noxious agents and of damaged tissue. Thus, the organs may be affected by inflammations that are diverse in terms of cause, symptoms and/or treatment. Inflammation can occur separately or in combination with a dermatological disorder, as described above. Hence, inflammations can also comprise a dermatological component.

In some embodiments, the dermatological disorders and/or inflammations involve the presence of Gram-positive bacteria, particularly bacteria of the genus Staphylococcus, particularly Staphylococcus aureus. Gram-positive bacteria, particularly bacteria of the genus Staphylococcus, particularly of the species Staphylococcus aureus may contribute to the development or progression of dermatological disorders. Gram-positive bacteria, preferably bacterium of the genus Staphylococcus or more preferably of the species Staphylococcus aureus may contribute to the development or progression of an inflammation. The term “Gram-positive bacteria” refers to bacteria in which the outer surface is typically the murein layer. Staphylococcus is a genus of Gram-positive bacteria, and can cause a wide variety of diseases in humans and animals through either toxin production or penetration. Staphylococci-related infection can range from mild and requiring no treatment to severe and potentially fatal. Over 30 different species of Staphylococcus can infect humans and non-human animals. Manifestations of staphylococcal infections usually depend on the type of infection, and the organism which causes it. Common types of infections include the following: skin infections (e.g., folliculitis, furuncles, impetigo, wound infections, scalded skin syndrome), soft-tissue infections (e.g., pyomyositis, septic bursitis, septic arthritis), mastitis, toxic shock syndrome, purpura fulminans, endocarditis, osteomyelitis, pneumonia, infections related to prosthetic devices (e.g., prosthetic joints and heart valves, vascular shunts, grafts, catheters), and urinary tract infection.

Individuals with suppressed immune systems (taking immune-suppressing medications or with immune deficiencies) are at increased risk of developing more serious infections. Staphylococcus species according to the present invention comprise: Staphylococcus arlettae, Staphylococcus aureus, Staphylococcus aureus NCTC 8325-4, Staphylococcus auricularis, Staphylococcus carnosus, Staphylococcus carprae, Staphylococcus chromogenes, Staphylococcus cohnii, Staphylococcus delphini, Staphylococcus dysgalactiae, Staphylococcus epidermidis, Staphylococcus equorum, Staphylococcus gallinarum, Staphylococcus hemolyticus, Staphylococcus hominis, Staphylococcus hyicus, Staphylococcus intermedius, Staphylococcus kloosii, Staphylococcus lentus, Staphylococcus lugdunensis, Staphylococcus muscae, Staphylococcus pasteuri, Staphylococcus pettenkoferi, Staphylococcus saprophyticus, Staphylococcus sciuri, Staphylococcus simulans, Staphylococcus warnerii, Staphylococcus xylosus. Preferably, the Staphylococcus species is Staphylococcus aureus.

In some of the presented embodiments, the recombinant polypeptide exhibits bacteriolytic activity in raw milk and/or serum. The term “bacteriolytic” is typically used to mean “bacterial cell wall degrading”, i.e. degrading the outer surface of the bacteria. Bacteriolytic activity leads to degradation, broking down, disintegration of the cell wall, or diminishing its integrity. Bacteriolytic activity may be an extreme form of cell-wall-degrading activity or the result of such an activity.

Preferably, the raw milk is raw cow's milk and the serum is preferably bovine or human serum. Raw cow's milk is non-processed and undiluted and comprises next to water, fat, lactose, various milk proteins also bacteria of the genus Lactococci, Lactobacilli and Leuconostoc. If a cow is a carrier of bacteria of the genus Staphylococcus or suffers from any form of mastitis, the milk also comprises bacteria of the genus Staphylococcus (staphylococci). The pH of cow's milk depends on various factors, i.a., the storage time and the presence of bacteria, including bacteria of the genus Staphylococcus. Regular cow's milk has a pH from about 6.0 to 7.0, cow's milk which comprises Staphylococcus has increased pH values compared to regular cow's milk. In that case, the pH values may range from 7.0 to 8.5. Hence, raw cow's milk is an emulsion with a highly specific environment, shaped by various factors including the presence of bacteria. Therefore, enzymes that exhibit activity in raw cow's milk need to be specifically targeted to these specific requirements. Similarly, fetal bovine serum stems from the blood of a bovine fetus and therefore, contains a complex array of protein components, in particular growth factors, hormones and amino acids. It also contains carbohydrates and lipids. Bovine serum albumin is the major component of fetal bovine serum. Therefore, enzymes that exhibit activity in raw cow's milk need to be specifically target to these specific requirements. Human serum corresponds to blood plasma without any coagulation factors. It comprises a complex array of protein components, in particular, growth factors, hormones and amino acids. It also contains carbohydrates and lipids.

The recombinant polypeptide according to the present invention may be formulated for topical administration. Topical refers to a mode of administration, and means that a material is administered by being applied to the skin or to the mucous membrane, which comprises one or more epithelial layers such as within the nipple, lactiferous canal, lactiferous sinus, eye, ear, nose or respiratory passage and genitals. The recombinant polypeptide for topical administration may be applied locally by external lubrication of the skin in a thin layer for prevention and/or treatment of mammary gland and/or udder tissue inflammations (including the teat/nipple, lactiferous canal, lactiferous ducts, and nipples), in particular for the treatment and/or prevention of mastitis. The recombinant polypeptide for topical administration may be applied locally by rinsing or dipping the affected area. Alternatively, the topical administration form can be applied by dropping it on or into the affected area. Preferably, the formulation is in the form of a cream, suspension, lotion, solution, ointment, gel, foam, transdermal patch, powder, paste, tape, pad, swab, wound dressing or compress.

In some embodiments, the recombinant polypeptide is formulated for intramammary delivery. The intramammary delivery refers to a form of delivery, in which the recombinant polypeptide is delivered via lactiferous ducts into the udder/teat/breast by injection, infusion or rinsing.

In some examples, the embodiment is referred to a recombinant polypeptide which is delivered to a teat or nipple, preferably by infusion, rinsing or dipping. The teat encompasses the nipple/teat, lactiferous ducts and lactiferous sinus. Infusion refers to the delivery of a composition comprising the recombinant polypeptide directly into the body site, where the composition comprising the recombinant polypeptide is required. Rinsing refers to cleansing by flushing with a composition comprising the recombinant polypeptide, thereby removing potential bacteria and simultaneously, specifically damaging the potential remainders of bacteria.

Preferred embodiments relate to the delivery of a recombinant polypeptide to a female human or non-human animal suffering from mastitis or at risk of developing mastitis, preferably a ruminant, more preferably a dairy cow. A female according to the present invention is an individual harboring mammary glands and can therefore, lactate. The non-human animal according to the present invention is a mammal. Ruminants comprise cattle, domesticated and wild bovines, goats, sheep, giraffes, deer, gazelles, and antelopes. Dairy cows comprise female cows which have the ability to lactate. At risk of developing mastitis refers to the risk of acquiring or developing the mastitis, i.e. causing at least one of the clinical symptoms to occur in a subject that may be exposed to, i.e. lactiferous canals/ducts and/or teat/nipple inflammation, in particular to mastitis, or predisposed to lactiferous canals/ducts and/or teat/nipple inflammation, in particular to the mastitis in advance of its onset. Alternatively, at risk of developing mastitis may also refer to the risk of acquiring or developing the mastitis, i.e. causing none of the clinical symptoms to occur in a subject that may be exposed to, if the subject is a carrier of bacteria of the genus Staphylococcus. Suffering from mastitis refers to the persistent, inflammatory reaction of the mammary gland and udder tissue, and is a major endemic disease of dairy cattle. It usually occurs when leukocytes are released into the mammary gland, as an immune response to bacterial invasion of the teat canal by variety of bacterial sources. Mastitis, in particular dairy animal mastitis, can be caused by many different bacterial species, one of the most common of which are staphylococci. Milk-secreting tissue and various ducts throughout the mammary glands are damaged due to toxins released by the bacteria.

Another aspect of the invention relates to administration of a veterinary composition comprising a recombinant polypeptide comprising (i) a LytM catalytic domain, comprising an amino acid sequence shown in SEQ ID NO: 1 or having at least 80% identity thereto, (ii) a lysostaphin cell wall binding domain, comprising an amino acid sequence shown in SEQ ID NO: 2 or having at least 80% identity thereto, and (iii) optionally, a peptide linker, linking the LytM catalytic domain and the lysostaphin cell wall binding domain, at least one excipient approved for use in pharmaceutical products. The veterinary composition comprising the recombinant polypeptide according to the invention may also comprise at least one excipient which modifies the release of the recombinant polypeptide and/or modifies the biodegradation of the recombinant polypeptide and/or stabilizes the at least one recombinant polypeptide during manufacturing of, storage and release. Moreover, the excipient may modify the solubility of the recombinant polypeptide. The excipient can, e.g., be a hydrophilic polymer, a sugar, a polyol, a surfactant, an antioxidant and/or a water-soluble salt or any other excipient known to achieve the abovementioned purposes.

Moreover, further excipients may be added to the veterinary composition comprising the polypeptide in order to optimize its chemical, physical and mechanical properties, in order to adapt it for the intended use. These additives include inter alia sodium benzoate, fatty acid esters or salts, trisodium phosphate, liquid paraffin, zinc oxide, calcium stearate, zinc stearate. The recombinant polypeptide formulated for topical administration may also contain other excipients, e.g. in order to improve the technical properties of the veterinary composition comprising the recombinant polypeptide, so that it may be easier to produce or in order to improve the stability. A suitable excipient approved for pharmaceutical use, for use in a veterinary composition according to the invention may be selected from the group consisting of fillers, diluents, disintegrants, glidants, pH-adjusting agents, viscosity adjusting agents, solubility increasing or decreasing agents, osmotically active agents and solvents.

The veterinary composition may further comprise excipients approved for pharmaceutical use, in order to increase the chemical stability. Preferred excipients are, e.g., chelating agents such as EDTA, disodium EDTA, deferoxamine, lipoic acid, glutathione, or antioxidants such as ascorbic acid palmitate, alpha-tocopherol, ubiquinol, 0-carotene, retinol and hydrophilic and/or lipophilic antioxidants.

Specific embodiments relate to the use of the veterinary composition for use in the prevention/and or treatment of dermatological disorders and/or inflammations. Dermatological disorders are disorders which affect the skin, which is made of layers of ectodermal tissue. The skin protects the underlying structures (e.g., muscles, bones, ligaments and internal organs) from contact with the outside environment or from injuries, including protection against pathogens such as bacteria. The skin may be affected by disorders that are diverse in terms of cause, presentation and/or treatment. The dermatological disorder may also be associated with inflammation. Inflammations are disorders which affect different organs by inflammation, i.e. a local response to cellular injury that is marked by any one or a combination of the following symptoms: capillary dilatation, leukocytic infiltration, redness, heat, and pain. Inflammation serves as a mechanism initiating the elimination of noxious agents and of damaged tissue. Thus, the organs may be affected by disorders that are diverse in terms of cause, symptoms and/or treatment. Inflammations can occur separately or in combination with a dermatological disorder, as described above. Hence, inflammations can also comprise a dermatological component.

Preferred embodiments relate to the veterinary composition, wherein the dermatological disorder and/or inflammation is selected from the group consisting of dermatitis, injuries and/or infections of a mucous membrane or of the skin, wounds, ulcers, and an inflammation of the udder, eye, ear, throat, sinuses or genitals. The mucous membrane according to this invention is the thin membrane that produces mucus and covers the inside surface of parts of the body. The mucous membrane comprises one or more epithelial layers such as within the teat, teat canal, teat cistern, eye, ear, nasal or respiratory passage and genitals. Ulcers are a discontinuity of the skin or a break in the skin or mucous membranes. Ulcers comprise pressure ulcers, genital ulcers, ulcerative dermatitis and anal fissures. Inflammation is a causative agent of ulcers.

In some embodiments, the veterinary composition comprising the recombinant polypeptide is formulated for intramammary administration. This administration refers to a form of delivery, wherein the recombinant polypeptide is delivered to a nipple or teat, by, for example, infusion, rinsing or dipping. The teat encompasses the teat/nipple, teat canal and teat cistern. Infusion refers to the delivery of a composition comprising the recombinant polypeptide directly into the body site, where the composition comprising the recombinant polypeptide is required. Rinsing refers to cleansing by flushing with a composition comprising the recombinant polypeptide, thereby removing potential bacteria and simultaneously, also the remainders of bacteria.

In yet another embodiment, the veterinary composition is provided for use in the treatment of mastitis in ruminants. Ruminants comprise cattle, domesticated and wild bovines, goats, sheep, giraffes, deer, gazelles, and antelopes. Dairy cows comprise female cows which have the ability to lactate. Suffering from mastitis refers to the persistent, inflammatory reaction of the mammary gland and udder tissue, and is a major endemic disease of dairy cattle. It usually occurs when leukocytes are released into the mammary gland, as an immune response to bacterial invasion of the teat canal by different bacteria. Mastitis, in particular in dairy cows, can be caused by many different bacterial species, the most common among them being Staphylococcus. Milk-secreting tissue and various ducts throughout the mammary glands are damaged due to toxins released by the bacteria.

The concentration of the recombinant polypeptide according to the invention, when used as a disinfectant, will typically be at least 10 nM, preferably at least 20 nM, more preferably at least 50 mM, and most preferably at least 100 nM. Typically, the concentration will be in the range from 10 nM to 1000 nM, preferably in the range from 20 nM to 500 nM, more preferably in the range from 20 nM to 200 nM. However, the effective concentration will depend on the formulation, the conditions applied, the environment etc., and can be even higher than 1 mM.

The concentration of the recombinant polypeptide, when used as an antibacterial agent within a pharmaceutical or veterinary composition, will typically be at least 10 nM, preferably at least 50 nM and more preferably at least 100 mM. Typically, the concentration will be in the range from 10 nM to 1000 nM, preferably in the range from 50 nM to 1000 nM, more preferably in the range from 100 nM to 1000 nM. However, the effective concentration will depend on the formulation and the conditions applied and can be even higher than 1 mM. This also applies to the use in intramammary administration in mastitis.

The recombinant polypeptide according to the invention can be used in combination with another active agent, such as an antibiotic or antiseptic agent. Examples of antibiotics that can be used in a combination therapy with the polypeptide according to the invention are vancomycin, erythromycin, daptomycin, and ciprofloxacin. Examples of antiseptics that can be used in combination with the recombinant polypeptide according to the invention are chlorhexidine, iodine, silver nanoparticles, and oxygen peroxide (up to 0.03%).

The recombinant polypeptide according to the invention can also be advantageously used in combination with detergents such as SDS (preferably up to 0.01%), polysorbate 20 (Tween 20; preferably up to 0.01%), polysorbate 80 (Tween 80; preferably up to 0.1%), octoxynol-9 (Triton X-100; preferably up to 0.1%), sorbitane monooleate (Span 80; preferably up to 0.1%), and common detergents as in dishwashing liquids and softeners.

The invention also relates to a cosmetic or care composition for external use, and thus improvement of the condition and appearance of the skin, especially in the case of skin infected with staphylococci, as well as acne skin by limiting the amount of staphylococci bacteria on the skin or eliminating their occurrence. The skin is subjected to various processes of destruction by dermatological diseases and undesirable cosmetic conditions, especially staphylococci and acne infections, which are often co-infected or secondarily infected with staphylococci. We have unexpectedly found further undisclosed use of the new recombinant polypeptide according to the invention in the form of a cosmetic or care composition for cosmetic, care and hygiene applications in humans and animals, for application on the skin to provide previously undisclosed treatment, preventive and care benefits. We have found that it is possible to effectively treat and prevent dermatological conditions and undesirable cosmetic conditions caused by staphylococci, particularly in acne co-infected with staphylococcus.

The composition according to the invention also contains a dermatologically/cosmetically acceptable carrier that acts as a diluent, dispersant or carrier for the active ingredients. The carrier may include materials commonly used in skin care products such as water, liquid or solid emollients, silicone oils, emulsifiers, solvents, humectants, thickeners, powders, propellants and the like. In addition to the active substance, which is the recombinant peptide according to the invention, other specific active substances beneficial to the skin, such as agents preventing the harmful effects of sunlight, agents with care and antibacterial properties, e.g., plant extracts or zinc, silver or copper ions, may also be included in the cosmetic or care composition. The carrier may also contain adjuvants such as fragrances, opacifiers, preservatives, dyes and buffers. Typical methods for making skin care products can be used to produce the composition for external use according to the invention. Oil-in-water or water-in-oil emulsions are preferred compositions. The compositions may be in the form of conventional care products such as cream, gel or lotion, ointment, lotion, powder or the like. It was shown that these active substances, carriers and additives used in the described compositions are inert to the enzymatic activity of the recombinant peptide according to the invention, and can be preferably used to support and/or enhance the enzymatic activity of the recombinant peptide according to the invention in the various applications mentioned herein.

### EXAMPLES

In the following examples, unless it was otherwise indicated, standard materials and methods used in the field were used, or the manufacturers' instructions for specific materials and methods were followed.

### Example 1: Cloning and Expression of Chimeric Enzymes

This example includes the preparation and delivery of two chimeric enzymes A and B that comprise the LytM catalytic domain, the lysostaphin cell wall binding domain of and a peptide linker.

### Cloning

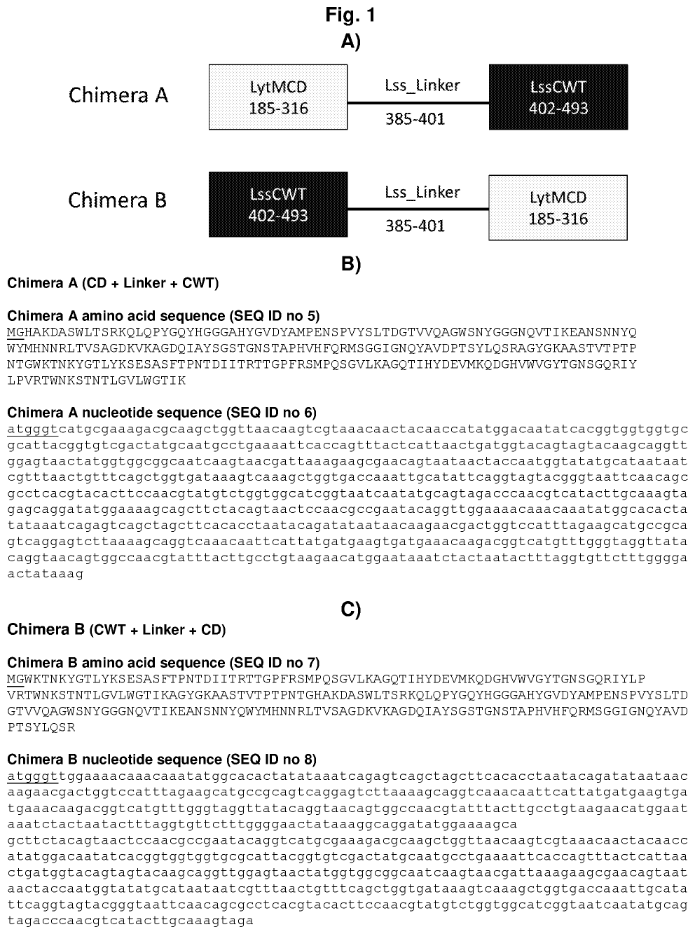

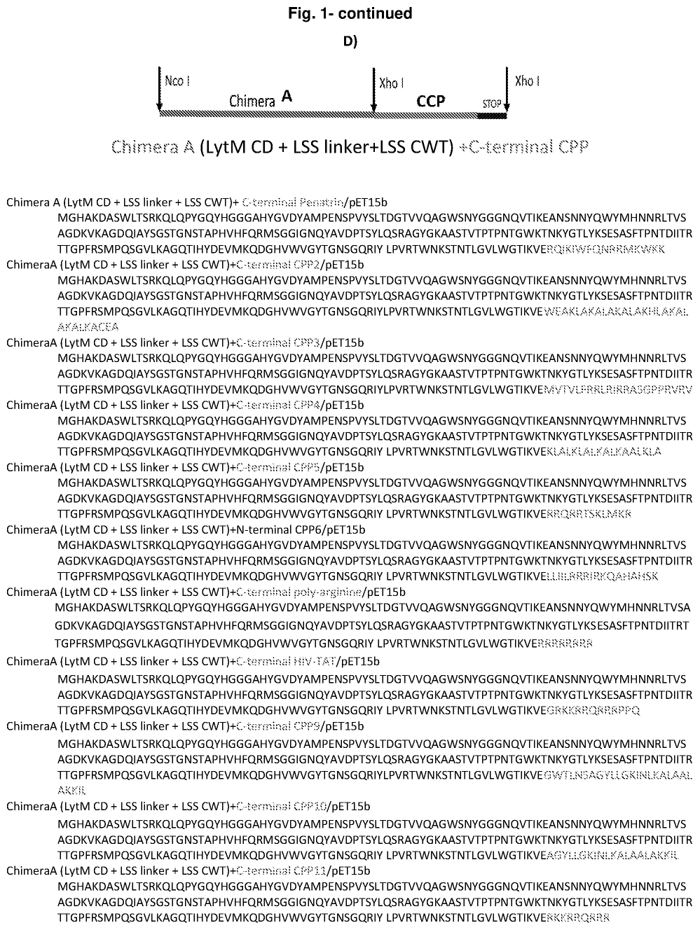

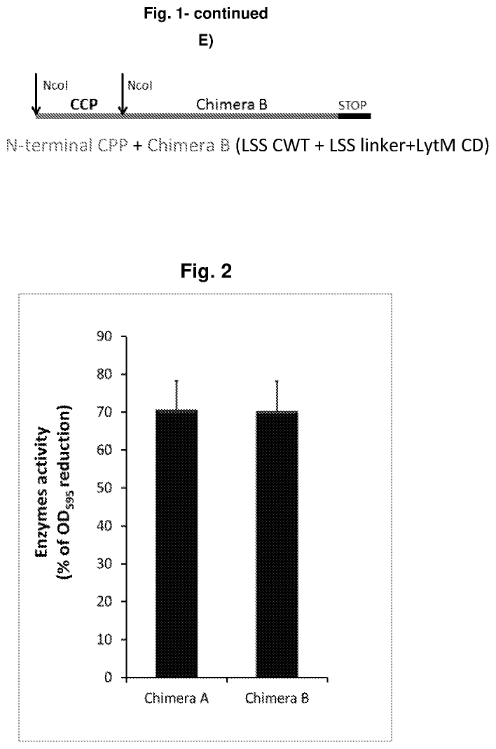

The catalytic domain (LytMCD, residues 185-316), the peptide linker (Lss_linker) and the lysostaphin cell wall binding domain (LssCWT) were ligated using overlap extension PCR in two different arrangements (FIG. 1). The primer is constructed so that it has a 5′ overhang, which is complementary to the end of another DNA molecule. After annealing and extension of the DNA, the obtained DNA fragments are multiplied by PCR using another pair of primes which are complementary to the target DNA ends. The obtained PCR products were purified and digested with NcoI and XhoI enzymes and ligated with similarly digested pET15b expression vector. E. coli TOP10 strain was transformed and after sequence verification, plasmids were transformed into E. coli BL21 (DE3) strain. Overexpression of proteins was induced by 1 mM IPTG and performed for about 4 hours at 25° C.

Mutagenesis Double mutants of the linker in Chimera A and Chimera B were produced by PCR-based site-directed mutagenesis with Phusion high-fidelity DNA polymerase (Thermo Fisher Scientific, Waltham, USA) using pET15b-ChimeraA/B/plasmids as a PCR template. The presence of the introduced mutations was confirmed by DNA sequencing.

### Attachment of Signal/Targeting Sequences

Constructs were produced, in which sequences that were signal/targeting sequences to the inside of an eukaryotic cell, were attached at the C-terminus of Chimera A and N-terminus of Chimera B. Signal sequences targeting the protein into the inside of a eukaryotic cell (Cell-Penetrating Peptides, CPPs) were cloned at the C-terminus of Chimera A into the XhoI restriction site or at the N-terminus of Chimera B into the NcoI restriction site. Attachment of the CCP sequences enables easy passage through the cell membrane and transport into the cell, e.g., into cytoplasm or cell organelles. DNA sequences of CPPs in the form of single-stranded oligonucleotides, designed so that the ends are recognized by the restriction enzyme, were hybridized, and then ligated with linearized vectors containing Chimera A or B sequences. The ligation mixture was transformed into E. coli TOP10 competent cells, then positive clones were sequenced, and E. coli BL21 (DE3) competent cells were transformed with the vectors with confirmed sequence. Protein overproduction was carried out in an auto-induction medium overnight at 25° C. Expression levels were verified on a polyacrylamide gel.

The sequences of the attached CPP signal sequences for which constructs with Chimera A and B were produced are given below in Table 2. It should be noted that other CPPs are known that can also be attached to the recombinant peptide according to the invention, to direct it into the cell.

TABLE 2

| SEQ ID No | Amino acid sequence | CPP name |
|---|---|---|
| 9 | RQIKIWFQNRRMKWKK | Penatrin |
| 10 | WEAKLAKALAKALAKHLAKALA | CPP 2 |
| | KALKACEA | |
| 11 | MVTVLFRRLRIRRASGPPRVRV | CPP 3 |
| 12 | KLALKLALKALKAALKLA | CPP 4 |
| 13 | RRQRRTSKLMKR | CPP 5 |
| 14 | LLIILRRRIRKQAHAHSK | CPP 6 |
| 15 | RRRRRRRR | poly-arginine |
| 16 | GRKKRRQRRRPPQ | HIV-TAT |
| 17 | GWTLNSAGYLLGKINLKALAAL | CPP 9 |
| | AKKIL | |
| 18 | AGYLLGKINLKALAALAKKIL | CPP 10 |
| 19 | RKKRRQRRR | CPP 11 |

The schematic structure of Chimera A with attached CPP, and the sequences of the recombinant peptides produced according to the invention with the CPP sequences attached at the C-terminus are shown in FIG. 1D. A schematic structure of Chimera B with CPP attached at the N-terminus is shown in FIG. 1E. The sequences of the produced recombinant peptides according to the invention, with CPP sequences attached at the N-terminus of Chimera B had the CPP sequences shown in Table 2.

### Protein Purification

The pellet from E. coli cultures containing the overexpressed proteins was resuspended in 20 mM of Tris-HCl pH 7.0, 1 M NaCl, 10% glycerol and sonicated. Cell lysate was dialyzed in 20 mM Tris-HCl, pH 7.0, 50 mM NaCl and purified by ion exchange chromatography using a NaCl gradient (SP Sepharose column, GE Healthcare). Further purification was performed by gel filtration on the Superdex 75 column (GE Healthcare, Uppsala, Sweden) in the buffer: 20 mM Tris-HCl pH 7.0, 200 mM NaCl, 10% glycerol.

Anti-staphylococcal activity of the purified chimeric enzymes was monitored by assays of turbidity reduction in bacterial cultures. Reference strains of S. aureus (NCTC 8325-4) were grown in Tryptic Soy Broth (TSB) medium at 37° C. The turbidity assays were performed in 96-well microtiter plates. Bacterial cells used in all experiments were collected in their exponential growth phase, suspended in TSB, washed, and resuspended in an appropriate buffer to obtain OD₅₉₅ of 1.0 (corresponding to 10⁸ CFU/ml). Cell lysis was monitored for 1 hour at room temperature. Readings were performed every 10 minutes after 5 seconds of shaking of the sample. Tests were carried out in 50 mM glycine buffer, pH 8.0, 100 mM NaCl. The lytic activity of both Chimeras (A and B) was nearly identical (FIG. 2). For this reason, further described in detail are only the experiments performed using Chimera A.

The produced Chimeras A and B with attached CPP signal sequences were characterized by a similar activity.

### Example 2: Chimera a is Active Against Staphylococcus Causing Mastitis

The aim of this example is the isolation of bacterial strains from milk of dairy cows suffering from mastitis and testing their susceptibility to an enzyme according to the invention—in this case Chimera A. Milk samples were taken from cows whose somatic cell counts (SSC) were increased.

The increased SCC is indicative of mastitis. Milk samples were taken from each quarter of the udder separately and plated on CHROMagar Mastitis (GrasoBiotech, Owidz, Poland). After 18 hours incubation at 37° C., single colonies of different morphologies were plated on fresh medium and grown for another 18 hours at 37° C. Isolated colonies were identified using the VITEK System (bioMerieux).

TABLE 3
Chimera A activity on bacteria causing bovine mastitis.
The enzyme activity was measured as reduction in
the turbidity of bacterial cells suspension.

| Strain | Chimera A activity |
|---|---|
| Staphylococcus aureus NCTC 8325-4 | +++ |
| Staphylococcus aureus | +++ |
| Staphylococcus equorum | ++ |
| Staphylococus sciuri | +++ |
| Staphylococcus simulans | +++ |
| Staphylococcus warnerii | +++ |
| Staphylococcus xylosus | ++ |
| Bacillus polymyxa | − |
| Corynebacterium spp. | − |
| Gardnerella vaginalis | − |
| Kocuria kristinae | − |

+++ represents an OD₅₉₅ reduction by 75-100%, ++ represents an OD₅₉₅ reduction by 50-75% and + represents an OD₅₉₅ reduction by 25-50% while − represents an OD₅₉₅ reduction by (0-25%). All the staphylococcal isolates were susceptible to treatment with Chimera A, while the non-staphylococcal species were not susceptible to Chimera A treatment.

### Example 3: Chimera a is Active in UHT Milk

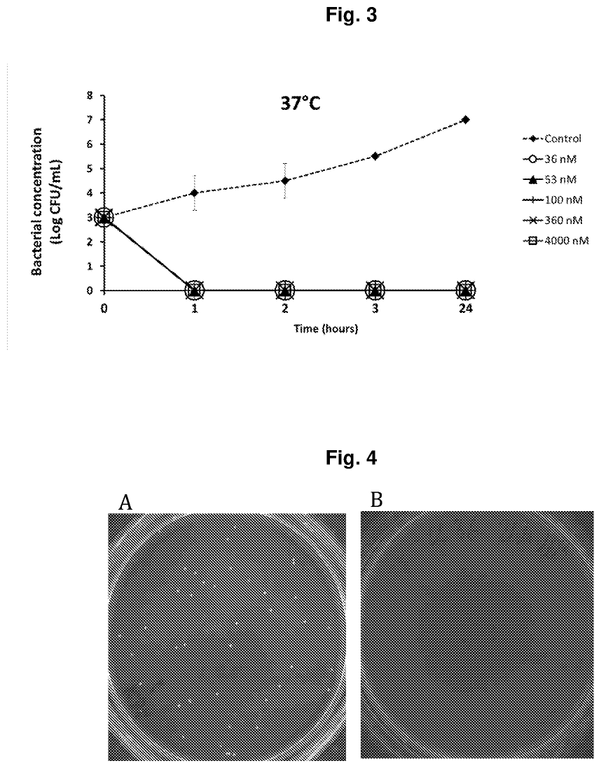

The aim of this example is to demonstrate the activity of the chimeric enzyme in UHT milk. The activity of Chimera A in UHT milk was estimated based on the reduction in the number of colonies (CFU) of Staphylococcus aureus in 1 ml after incubation in milk, in the presence of the tested enzymes. Staphylococcus aureus NCTC 8325-4 strain was grown to OD₅₉₅ of 0.6 in TSB at 37° C. with mild agitation. The activity of Chimera A at 37° C. was determined for the initial number of cells at a concentration of 10³ CFU/ml and for various enzyme concentrations (36-4000 nM). Even at concentrations of Chimera A as low as 36 nM, S. aureus was completely eradicated from milk to an undetectable level within 1 hour of incubation (FIG. 3 and FIG. 4).

UHT milk was inoculated with S. aureus in the amount of 10⁶ CFU/ml and incubated at 4° C. or room temperature in the presence of 100 nM of the enzyme. Samples were taken after 2 and 24 hours of incubation and the bacterial count was determined by serial dilution and plating on TSB-agar plates. Plates were incubated at 37° C. for 18 hours. Milk incubated without enzymes was used as a negative control. Experiments were performed in three independent repeats.

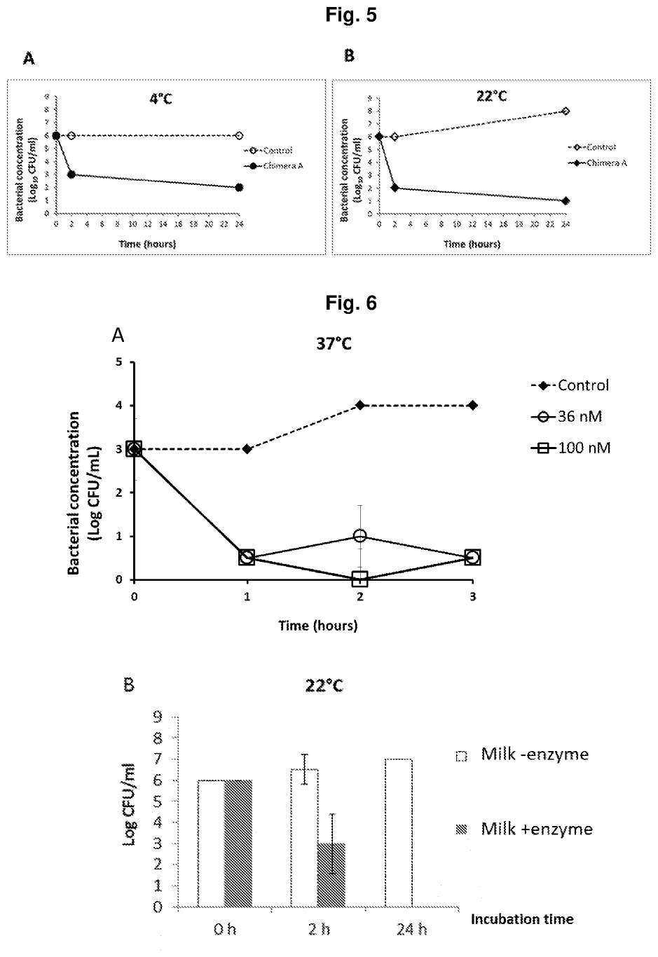

Chimera A eliminated S. aureus from UHT milk, both at 4° C. and 22° C. (FIG. 5), and was much more effective at room temperature (reduction of CFU/ml by 8 orders of magnitude). The initial number of staphylococcal cells (1 million/ml) was reduced by at least 99.9% in just 2 hours of incubation with the enzyme.

### Example 4: Chimera a is Active in Raw Cow's Milk

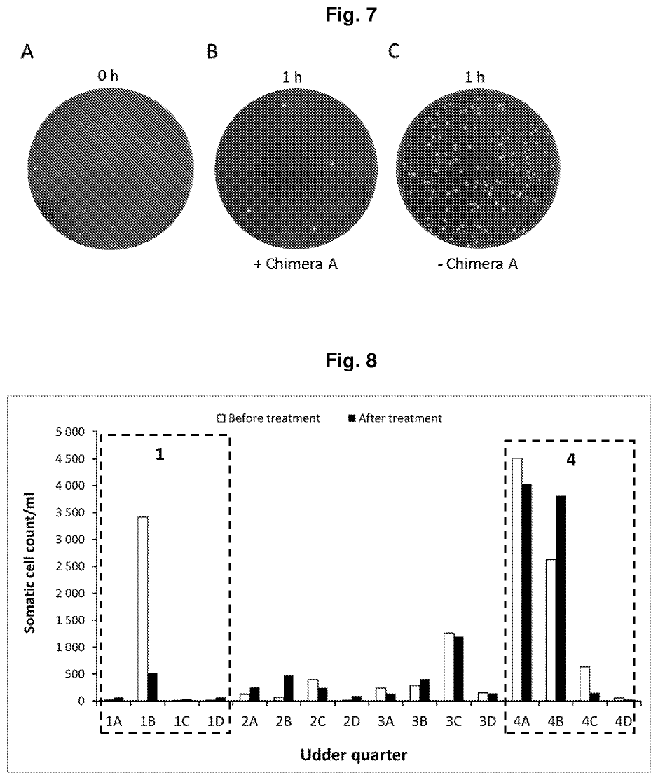

The aim of this example is to demonstrate the activity of the chimeric enzyme in raw cow's milk. In order to determine the effect of raw milk on the activity of the chimeric enzyme, raw cow's milk with the addition of S. aureus was incubated in the presence of 36 and 100 nM of Chimera A for 3 hours. The activity of Chimera A in raw cow's milk was determined as a decrease in CFU/ml log over time. Raw milk was inoculated with the NCTC 8325-4 S. aureus strain, cultured to an OD₅₉₅ of 0.6 in TSB at 37° C. with mild agitation, at a concentration of 10³ CFU/ml. The inoculated milk was incubated in the presence of various concentrations of Chimera A at 37° C. Samples were taken after 0, 1, 2 and 3 hours, plated on TSB-agar plates, which were incubated at 37° C. for 18 hours. The surviving cells were counted and CFU/ml log decrease was estimated. The sample incubated without the enzyme served as negative control. Experiments were repeated three times. Chimera A eliminated over 99% of the initial cells number within 1 hour (FIG. 6A and FIG. 7). In subsequent tests, Chimeras' ability to eliminate greater number bacteria was checked. 10⁶ CFU/ml of S. aureus (strain NCTC 8325-4) cells, present in milk were treated with 100 nM of Chimera A at 22° C. for 24 hours. After just two hours, the number of bacterial cells decreased to 10³ CFU/ml and after 24 hours, 100% of the bacterial cells were eliminated (FIG. 6B).

### Example 5: Chimera a Causes a Decrease in Somatic Cell Counts in Milk Obtained from Mastitis-Affected Cows

The aim of this example is to demonstrate that somatic cell counts decreased in milk samples from mastitis-affected cows after treatment with Chimera A. Four cows belonging to an experimental herd of dairy cattle stemming from an experimental farm were selected for the experiment. The selection of the cows was based on the somatic cell count over the past 8 months; the selected cows had an increased somatic cell count in at least one quarter of their udder, which indicated different stages of mastitis development. Immediately prior to the start of the experiment, the somatic cell counts were determined in milk collected from each quarter and the obtained milk sample underwent microbiological analysis. Bacteria were cultured and the species were identified as described in Example 1. The same analysis was performed for milk collected at the end of the experiment.

The enzyme solution contained 20 mM of Tris-HCl at pH 7.4, 100 mM NaCl, 10% glycerol and 100 nM of Chimera A. Immediately after milking, each quarter was washed with the enzyme solution. This treatment was repeated twice a day, for seven days.

In the case of cow 1, the initial somatic cell count was almost 3.5 million in the milk obtained from quarter B, which was representative for acute mastitis. In the other quarters (A, C, D), the somatic cell count did not exceed 20,000 which indicates lack of inflammation (FIG. 8). Microbiological tests of milk samples demonstrated that the somatic cell count and therefore, inflammation of quarter B was caused by Staphylococcus. After seven days of udder treatment with Chimera A solution, the somatic cell count of the milk obtained from quarter B was reduced highly significant to slightly above 500,000. The somatic cell count of the other quarter (A, C, D) changed slightly and did not exceed 100,000, which is a standard level for healthy animals and indicates the absence of infection. The staphylococcal species isolated from the infected quarter were identified as S. xylosus. In the in vitro Chimera A susceptibility tests, this staphylococcal strain proved to be sensitive to the used chimeric enzyme (Table 3).

A similar effect was observed in cow 4 whose somatic cell counts indicated acute inflammation in two teats (udder quarters): teat A and B (somatic cell count above 4.5 million and 2.6 million, respectively) and a slightly less severe mastitis in teat C (SCC) over 600,000. The fourth quarter (D) did not show any signs of inflammation (somatic cell count of 54,000). The microbiological analysis of the milk samples revealed the presence of Staphylococcus only in teat C. Solely the associated quarter of teat C showed a significant reduction of the somatic cell count from above 623,000 to 150,000 after treatment with the chimeric enzyme solution for one week. In all other quarters, no significant changes in somatic cell count were observed, also no pathogenic staphylococcal species were detected in milk samples taken from these quarters.

### Example 6: Chimera a is Active in Serum

The aim of this example is to demonstrate the activity of the chimeric enzyme in serum. The lytic activity of 100 nM solution of the Chimera A enzyme was tested in turbidity reduction assays in the presence of fetal bovine serum. The test was performed in 96-well microtiter plates.

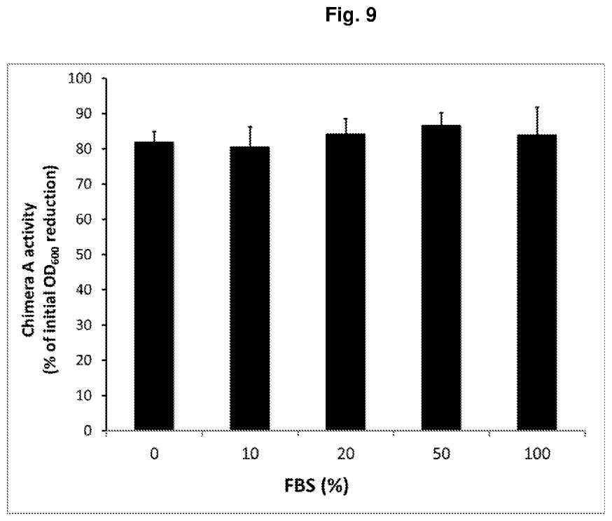

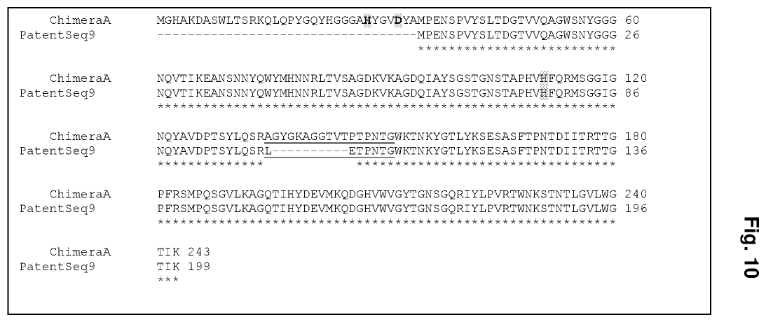

Staphylococcus aureus NCTC 8325-4 reference strain was cultured to logarithmic growth phase, washed and suspended in 50 mM glycine buffer, pH 8.0, 100 mM NaCl to obtain OD₅₉₅ of 1.0. Tests were carried out in 50 mM glycine buffer, pH 8.0, 100 mM NaCl with the addition of fetal bovine serum (FBS) at the final concentration from 0 to 100%. The decrease in OD₅₉₅ was monitored for 1 hour at room temperature, with readings every 10 minutes, after 5 seconds of shaking. The positive control did not contain FBS. The activity of the chimeric enzyme was not altered in the presence of FBS, even at the concentration of 100% (FIG. 9).

In order to confirm that the chimeric enzyme is as active in 100% serum as in optimal conditions, the number of live cells was determined and showed that the enzyme eliminated more than 99.99% (5 orders of magnitude decrease) of staphylococcal cells within 2 hours (Table 4). The same result was obtained for the activity of the chimeric enzyme in glycine buffer.

TABLE 4
The activity of the chimeric enzyme A in the presence of 100%
of fetal bovine serum (FBS). The activity of 100 nM of the
enzyme was estimated as a log10 decrease of CFU/ml within 2 h at
room temperature (decrease in log₁₀ CFU/ml).

| | Bacterial concentration | | CFU/ml log₁₀ |
|---|---|---|---|
| | (CFU/ml) Mean ± SD | | decrease after |
| | 0 h | 2 h | 2 h |
| Buffer (Control) | 2.71 ± 0.45 × 10⁸ | 2.25 ± 0.43 × 10⁸ | 0 log |
| Buffer + | 2.71 ± 0.45 × 10⁸ | 2.40 ± 1.41 × 10³ | 5 log |
| Chimera A | | | |
| FBS (Control) | 2.65 ± 0.43 × 10⁸ | 5.05 ± 0.28 × 10⁸ | 0 log |
| FBS + Chimera A | 2.65 ± 0.43 × 10⁸ | 5.40 ± 0.28 × 10³ | 5 log |

### Example 7. Comparative Examples

### A. Comparison of Chimeras' Activity

Verbree et al. have published the results of analyzes of about 170 different lysins of phage origin and various modified chimeras tested for activity in milk (Verbree, Datwyler et al., 2018). Only 8 of the 170 enzymes tested showed limited activity in pasteurized milk, but none of them was effective in removing staphylococcus cells from raw milk. A slight decrease in the number of staphylococcus cells in raw milk was observed only in mixtures of different bacteriolytic enzymes administered simultaneously and at very high concentration. None of the tested enzymes was effective in eliminating large numbers of bacterial cells at low enzyme concentrations.

We compared the activities of the chimeras according to the invention—Chimera A—by conducting experiments with them under identical conditions and concentrations in which the chimeras described in Verbree, Datwyler et al. (2018) were tested. Table 5 shows the obtained comparative results presented as orders of magnitude of the number of cells, and orders of decrease in the number of cells (A) after treatment with appropriate enzymes.

TABLE 5
Comparison of the effects of the enzymes according to the invention with the results
of tests of other chimeras presented in Verbree, Datwyler et al., 2018.

| | | | | Cell count after three hours of treatment with the | | | |
|---|---|---|---|---|---|---|---|
| Test conditions | | | | enzyme log₁₀ | | | |
| | | Control cell count | | | | | |
| | Enzyme | log₁₀ | | Results Verbree et al. | | Results Chimera A | |
| Milk | concentration | T₀ | T_{3c} | T_{3E} | Δ | T_{3E} | Δ |
| UHT | 36 nM | 3 | 5 | 4-5 | 0-1 | 0 | 5 |
| UHT | 360 nM | 3 | 5 | 0-4 | 1-5 | 0 | 5 |
| UHT | 4000 nM | 3 | 5 | 3-0 | 2-5 | 0 | 5 |
| RAW 100% | 36 nM | 3 | 4 | — | — | 0 | 4 |
| RAW 100% | 320 nM | 3 | 4 | 4-1 | 1-3 | — | |

Number of bacteria in orders of magnitude.
T₀ - initial; T_{3C} - after 3 hours without the enzyme, T_{3E} - after 3 hours with the enzyme, Δ - decrease in the number of bacteria after 3 hours of treatment with the enzyme, expressed in orders of magnitude.
RAW- raw milk.

It was shown that Chimeras according to the invention exhibit many times higher activity, even when used at very low concentrations. The high activity of the Chimeras according to the invention was observed not only in UHT milk; it was also found that these Chimeras are very effective in removing staphylococcus cells from raw milk, a property not shared by any chimera known in the state of the art.

B. No activity of a chimera known in the state of the art.

In the patent application WO2007/130655 from Ganagagen, chimeric enzymes consisting of the active LytMCD domain and the lysostaphin cell wall binding domain were described. Alignment of the amino acid sequence of Chimera A according to the invention to the chimera shown in Seq Id No 9 in the WO2007/130655 publication is shown in FIG. 10.

As can be seen from the presented alignment, the chimera sequence Seq ID No 9 known in the state of the art includes only a part of the peptide building catalytic domain, and it lacks 34 amino acids at the N-terminus, including two coordinating zinc residues H210 and D214, which have been experimentally shown to be necessary for the operation of the LytM catalytic domain (Odintsov, Sabala et al., 2004) and other members of this family, such as Ale-1 (Fujiwara, Aoki et al., 2005) and lysostaphin (Bardelang, Vankemmelbeke et al., 2009). The absence of each of these residues separately, especially both at the same time, blocks the catalytic activity of the enzyme determining its antibacterial properties. A construct with an incomplete active center cannot carry out the cleavage reaction of peptide bonds leading to cell wall disintegration and thus lysis of bacterial cells. It was demonstrated that the chimeras known in the state of the art are catalytically inactive.

### Example 8. Comparison of Chimera B Activity with and without CPP Sequences Under Different Environmental Conditions

Lytic activity studies against S. aureus under various environmental conditions were carried out for the Chimera B constructs produced according to Example 1 with and without CPP signal sequences.

The comparison of the activity was carried out for: i) Chimera B, ii) Chimera B with CPP (signal sequence RQIKIWFQNRRMKWKK (SEQ ID No. 9) attached at the N-terminus). The activity of the enzymes was compared under the following conditions.

### 8.1. Activity in Glycine Buffer

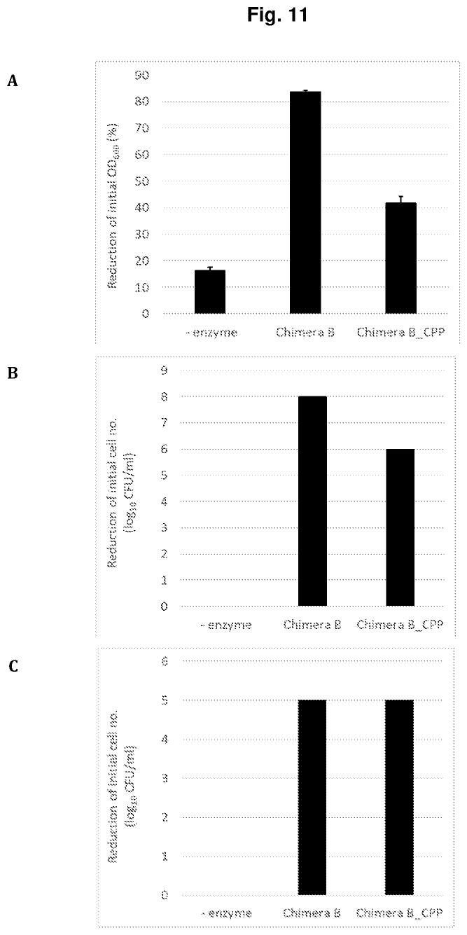

The lytic activity of the purified chimeric enzymes was monitored in the test of turbidity reduction of bacterial suspensions. Reference strain of S. aureus (NCTC 8325-4) was grown in Tryptic Soy Broth (TSB) medium at 37° C. and bacterial cells were collected at exponential growth phase, washed, and suspended in 50 mM glycine, pH 8.0, 100 mM NaCl buffer to OD₅₉₅ 1.0 (corresponding to 10⁸ CFU/ml). The activity of 500 nM of enzymes, observed as a decrease in OD₅₉₅, was monitored for 1 hour at room temperature and the values shown in the graph present the decrease in starting OD₅₉₅ after 1 hour (mean±SD). The negative control was a sample incubated without the enzyme. The experiment was repeated 3 times in triplicates. The obtained mean results are shown in FIG. 11A.

### 8.2. Activity in Serum

The lytic activity of the chimeric enzymes was measured as a percentage of the reduction of the initial number of cells that were used to inoculate fetal bovine serum. The reference strain of S. aureus (NCTC 8325-4) was grown in Tryptic Soy Broth (TSB) medium at 37° C., bacterial cells were collected at exponential growth phase. The serum was inoculated with 10⁸ CFU/ml and incubated with 500 nM of the specified enzyme for 3 hours at 37° C. The number of bacterial cells was determined in samples collected after 3 hours by serial dilutions which were plated on TSB-agar plates. The plates were incubated at 37° C. for 18 hours and the result was presented as the log₁₀ decrease of the initial CFU/ml. The negative control was a sample incubated without the enzyme. The experiment was repeated 3 times in triplicates. The obtained mean results are shown in FIG. 11B.

### 8.3. Activity in UHT Milk

The lytic activity of the chimeric enzymes was measured as a percentage of the reduction of the initial number of cells that were used to inoculate 50% milk (diluted with 50 mM glycine, pH 8.0, 100 mM NaCl). The reference strain of S. aureus (NCTC 8325-4) was grown in Tryptic Soy Broth (TSB) medium at 37° C. and the bacterial cells were collected at exponential growth phase. The serum was inoculated with 10⁶ CFU/ml and incubated with 1 μM of the appropriate enzyme for 3 hours at 37° C. The number of bacterial cells was determined in samples collected after 3 hours by serial dilutions which were plated on TSB-agar plates. The plates were incubated at 37° C. for 18 hours and the result was presented as the log₁₀ decrease of the initial CFU/ml. The negative control was a sample incubated without the enzyme. The experiment was repeated 3 times in triplicates. The obtained mean results are shown in FIG. 11C. It was shown that the binding of the CPP domain does not adversely affect the activity of Chimera A, and particularly, its activity in milk and serum.

### Example 9. The Use of the Recombinant Polypeptides According to the Invention for the In Vivo Treatment of Infected Wounds

9.1. Preparation of a Composition of Hydrogel Wound Dressings with Chimera A and B

The commercially available wound dressings (AquaGel, KIKGEL) were cut into 1 cm² pieces and spotted with a standard PBS buffer solution (negative control) or the protein solution of the invention Chimera A or B in PBS buffer (test sample). The solutions were dropped directly onto the hydrogel pieces: for the test sample, 100 nM enzyme solution at a volume of 100 μl; for the control, 100 μl of PBS buffer) and left until the solution was absorbed. The hydrogels prepared in this way were stored at 4° C. until they were applied to the wounds.

9.2 In vivo antibacterial and anti-inflammatory effect of the hydrogel wound dressing with the recombinant polypeptide according to the invention

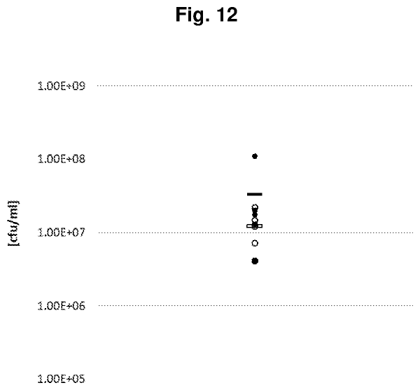

The overnight culture of the S. aureus FDA485 strain was centrifuged and resuspended in sterile PBS buffer. Bacterial cell count was adjusted to 1 in McFerland scale (corresponding to 1.61×10⁸ CFU/ml). Five female BL/6 mice were tested in each experimental variant. Surface wounds were generated by needle scratch, 50 μl of a bacterial suspension was applied directly onto the scratch and covered with a sterile cotton linter (ROTH). The infection developed for 24 h. After this time, the cotton linter was removed and a hydrogel wound dressing (without a Chimera according to the invention—negative control or with the test sample—Chimera A or B) was applied to the infected wounds and placed directly on the wound. Every 12 hours the wound dressing was changed to a new one of the same type. 72 hours after the infection of the wound, all mice were sacrificed. The wound along with the surrounding skin was homogenized and suspended in 3 ml of PBS. Serial dilutions were made and plated on TSB-agar plates. Two serial dilutions were made for each variant. To calculate the mean value of bacterial infection, the grown S. aureus colonies were counted after the plates were incubated overnight at 37° C. The obtained results were averaged for the results from at least three test plates. The obtained mean results are shown in FIG. 12.

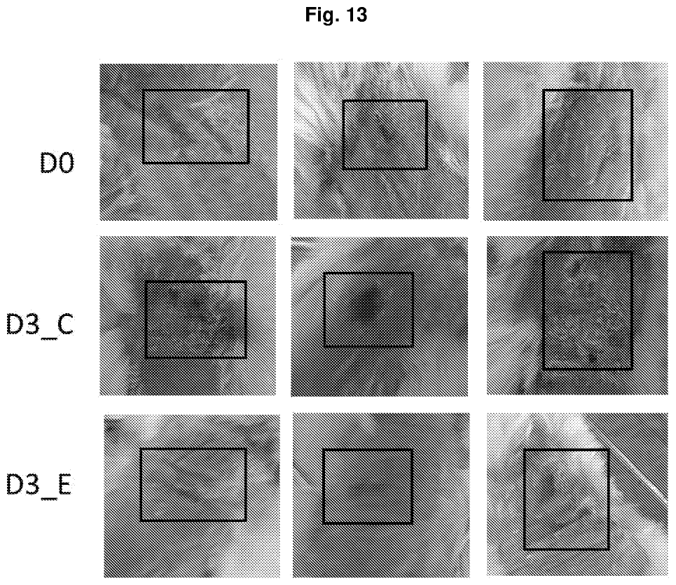

Photos of wounds during the experiment were presented in FIG. 13. The top panel DO shows an example of the appearance of wounds on three mice at the beginning of the experiment (day 0). Slightly reddened wounds on the surface of the skin with no hair are visible. The middle panel (D3_C) shows exemplary photos of the skin of control mice with infected wounds on which hydrogel wound dressings without enzyme were applied for three days. The development of a bacterial infection and inflammation caused by it, manifested by reddening of the skin around the wounds can be observed. The lower panel (D3_E) shows examples of the previously infected wounds onto which the enzyme-Chimera A hydrogel wound dressings according to the invention were applied for three days. These wounds look very similar to the wounds from the beginning of the experiment (top panel DO). Wounds healing and inflammation subsiding due to the activity of the Chimera according to the invention contained in the dressing is clearly visible. The inflammation observed in the control group (middle panel D3_C) has resolved, and there is no clearly visible redness around the wounds.

The performed in vivo tests showed a significant decrease in the number of S. aureus bacteria in wounds treated with the Chimera dressing according to the invention. There was a 63% reduction in the number of bacterial cells (reduction by 2.1×10⁷ CFU/ml) in the treated wound compared to the untreated wound. For statistical verification, a two-way ANOVA analysis was performed on the values after logarithmic transformation. The difference between the negative control and the sample is statistically significant (p<0.01). The obtained mean results are shown in FIG. 12.

The recombinant polypeptides according to the invention show antibacterial activity against S. aureus when delivered to an infected wound via a hydrogel, as well as an anti-inflammatory activity.

The recombinant polypeptides according to the invention show in vivo activity as a medicine against Gram-positive bacteria, particularly staphylococci, and are particularly useful for the treatment of S. aureus infections.

### LITERATURE

Bardelang, P., M. Vankemmelbeke, Y. Zhang, H. Jarvis, E. Antoniadou, S. Rochette, N. R. Thomas, C. N. Penfold and R. James (2009). “Design of a polypeptide FRET substrate that facilitates study of the antimicrobial protease lysostaphin.” Biochem J 418(3): 615-624.
Bowler et al. (2001). “Wound microbiology and associated approaches to wound management.” Clin Microbiol Rev 14(2): 244-269.
Brusselaers et al. (2010). “Severe burn injury in Europe: a systematic review of the incidence, etiology, morbidity, and mortality.” Crit Care 14(5): R188.
Fujiwara, T., S. Aoki, H. Komatsuzawa, T. Nishida, M. Ohara, H. Suginaka and M. Sugai (2005). “Mutation analysis of the histidine residues in the glycylglycine endopeptidase ALE-1.” J Bacteriol 187(2): 480-487.
Hicks et al. (2014). “Trends and determinants of costs associated with the inpatient care of diabetic foot ulcers.” J Vase Surg 60(5): 1247-1254, 1254.e1241-1242.
Hop et al. (2014). “Costs of burn care: a systematic review.” Wound Repair Regen 22(4): 436-450.
Jagielska, E., O. Chojnacka and I. Sabala (2016). “LytM fusion with SH3b-like domain restore its activity in physiological conditions.”
Karthikesalingam et al. (2010). “A systematic review of scoring systems for diabetic foot ulcers.” Diabetic Medicine 27(5): 544-549.
Kruse and Edelman (2006). “Evaluation and Treatment of Diabetic Foot Ulcers.” Clinical Diabetes 24(2): 91-93.
Malinowski and Smulski (2007). “Prevalence and prophylaxis of intramammary infections and mastitis in pregnant heifers.” Zycie Weterynaryjne 82(6): 476-482.
Odintsov, S. G., I. Sabala, M. Marcyjaniak and M. Bochtler (2004). “Latent LytM at 1.3A resolution.” J Mol Biol 335(3): 775-785.
Osipovitch, D. C. and K. E. Griswold (2015). “Fusion with a cell wall binding domain renders autolysin LytM a potent anti-Staphylococcus aureus agent.” FEMS Microbiol Lett 362(2): 1-7.
Osipovitch D C, Therrien S, Griswold K E (2015). “Discovery of novel S. aureus autolysins and molecular engineering to enhance bacteriolytic activity”. Appl Microbiol Biotechnol. 2015 August; 99(15): 6315-26.
Rodriguez-Rubio, L., B. Martinez, D. M. Donovan, P. Garcia and A. Rodriguez (2013). “Potential of the virion-associated peptidoglycan hydrolase HydH5 and its derivative fusion proteins in milk biopreservation.” PLoS One 8(1): e54828.
Verbree, C. T., S. M. Datwyler, S. Meile, F. Eichenseher, D. M. Donovan, M. J. Loessner and M. Schmelcher (2018). “Corrected and Republished from: Identification of Peptidoglycan Hydrolase Constructs with Synergistic Staphylolytic Activity in Cow's Milk.” Appl Environ Microbiol 84(1).

1. A recombinant polypeptide comprising
i. a LytM catalytic domain (LytMCD), comprising an amino acid sequence shown in SEQ ID NO: 1 or having at least 80% identity thereto, which retains the bacteriolytic activity against Staphylococcus aureus and wherein the zinc-coordinating amino acids in the active center are not changed and are His in position 26, Asp in position 30, His in position 109 of the SEQ ID NO: 1;
ii. a lysostaphin cell wall binding domain (LssCWT), binding the cell wall of bacteria, comprising an amino acid sequence shown in SEQ ID NO: 2 or having at least 80% identity thereto; and
wherein the recombinant polypeptide optionally has a peptide linker linking the LytM catalytic domain and the lysostaphin cell wall binding domain,
wherein the recombinant polypeptide its N or C terminus, has an attached Cell-Penetrating Peptide (CPP) signal sequence directing to the inside of an eukaryotic cell, and
wherein the recombinant polypeptide is for use in a treatment of a disease and/or condition caused by a bacterium of a genus Staphylococcus.

2. The recombinant polypeptide for use according to claim 1, wherein the LytM catalytic domain consists of the amino acid sequence shown in SEQ ID NO: 1.

3. The recombinant polypeptide for use according to claim 1, wherein the lysostaphin cell wall binding domain consists of the amino acid sequence shown in SEQ ID NO: 2.

4. The recombinant polypeptide for use according to claim 1, wherein the peptide linker, linking the LytM catalytic domain and the lysostaphin cell wall binding domain, comprises the amino acid sequence shown in SEQ ID NO: 3 or SEQ ID NO: 4, or consists of the sequence SEQ ID NO: 3.

5. The recombinant polypeptide for use according to claim 1, characterized in that it comprises the sequence SEQ ID NO: 5 or is encoded by a nucleotide sequence comprising SEQ ID NO: 6, or consists of SEQ ID NO: 5.

6. The recombinant polypeptide for use according to claim 1, characterized in that it comprises the sequence SEQ ID NO: 7 or is encoded by a nucleotide sequence comprising SEQ ID NO: 8, or consists of SEQ ID NO: 7.

7. The recombinant polypeptide for use according to claim 1, wherein the disease and/or condition is selected from the group consisting of acne, pimples, dermatitis, preferably atopic dermatitis, infections and/or injuries of a mucous membrane or of the skin, wounds, diabetic foot, ulcers, burns, and an inflammation of the mammary gland, inflammation of the udder, eye, ear, throat, sinuses or genitals.

8. (canceled)

9. The recombinant polypeptide for use according to claim 1, wherein the recombinant polypeptide is formulated for topical administration, preferably in the form of a cream, suspension, lotion, solution, ointment, gel, foam, transdermal patch, powder, paste, suspension, tape, pad, swab, wound dressing or compress.

10. The recombinant polypeptide for use according to claim 1, wherein the recombinant polypeptide is formulated for intramammary delivery, preferably the polypeptide is delivered to a nipple, teat, udder, preferably by infusion, injection rinsing or dipping.

11. The recombinant polypeptide for use according to claim 10, wherein the recombinant polypeptide is delivered to a female human or non-human animal suffering from mastitis or at risk of developing mastitis, preferably a ruminant, more preferably a dairy cow.

12. A pharmaceutical or veterinary composition comprising the recombinant polypeptide of claim 1 and a pharmaceutically or veterinary acceptable excipient for use as medicine in the treatment of the disease and/or condition caused by bacterium of the genus Staphylococcus.

13-17. (canceled)

18. The pharmaceutical or veterinary composition according to claim 12 for use in the disease and/or condition selected from the group consisting of dermatitis, injuries and/or infections of a mucous membrane or of the skin, wounds, ulcers, inflammation of the mammary gland, inflammation of an eye, ear, throat, sinuses or genitals, mastitis in ruminants.

19-20. (canceled)

21. The recombinant polypeptide comprising:
(i) a LytM catalytic domain (LytMCD), comprising an amino acid sequence shown in SEQ ID NO: 1 or having at least 80% identity thereto which retains the bacteriolytic activity against Staphylococcus aureus, and wherein the zinc-coordinating amino acids in the active center are not changed and are His in position 26, Asp in position 30, His in position 109 of the SEQ ID NO: 1;
(ii) lysostaphin cell wall binding domain (LssCWT), binding the cell wall of bacteria, comprising an amino acid sequence shown in SEQ ID NO: 2 or having at least 80% identity thereto; and
wherein the recombinant polypeptide optionally has a peptide linker linking the LytM catalytic domain and the lysostaphin cell wall binding domain; and
wherein the recombinant polypeptide from the LssCWT domain side, at it's N or C terminus, has an attached CPP signal sequence directing to the inside of a eukaryotic cell;
wherein the recombinant polypeptide is for use as a disinfectant, in-vitro antibacterial agent, in-vitro active agent in a cosmetic, care or hygienic composition for humans or animals against bacterium of a genus Staphylococcus, preferably.

22-26. (canceled)

27. The recombinant polypeptide according to claim 21, wherein the polypeptide is used as the disinfectant, and wherein the disinfectant is used for disinfecting surfaces, appliances and equipment, furniture, floors, particularly in hospitals, medical and dental offices, sports facilities, production lines, food factories and processing plants, kitchens and bathrooms.

28. The recombinant polypeptide according to claim 21, wherein the polypeptide is used as an antibacterial agent in milk, serum, preferably in whole milk.

29. A cosmetic or care composition for cosmetic, care and hygienic use in humans or animals, wherein it comprises a recombinant polypeptide comprising
(i) a LytM catalytic domain (LytMCD), comprising an amino acid sequence shown in SEQ ID NO: 1 or having at least 80% identity thereto which retains the bacteriolytic activity against Staphylococcus aureus, and wherein the zinc-coordinating amino acids in the active center are not changed and are His in position 26, Asp in position 30, His in position 109 of the SEQ ID NO: 1;
(ii) a lysostaphin cell wall binding domain (LssCWT), binding the cell wall of bacteria comprising an amino acid sequence shown in SEQ ID NO: 2 or having at least 80% identity thereto, and
wherein the recombinant polypeptide optionally has a peptide linker, linking the LytM catalytic domain and the lysostaphin cell wall binding domain,
wherein the recombinant polypeptide, from the LssCWT domain side, at it's N or C terminus has an attached CPP signal sequence directing to the inside of a eukaryotic cell,
wherein the composition additionally comprises at least one carrier approved for care and hygienic use in humans or animals, and is intended for external use, and
wherein the recombinant polypeptide is used to limit the occurrence or to remove bacteria of a genus Staphylococcus.

30-34. (canceled)

35. A method for in-vivo treatment of mastitis or prevention of developing mastitis caused by a bacterium of the genus Staphylococcus with a recombinant polypeptide comprising
(i) a LytM catalytic domain (LytMCD), comprising an amino acid sequence shown in SEQ ID NO: 1 or having at least 80% identity thereto which retains the bacteriolytic activity against Staphylococcus aureus, and wherein the zinc-coordinating amino acids in the active center are not changed and are His in position 26, Asp in position 30, His in position 109 of the SEQ ID NO: 1;
(ii) a lysostaphin cell wall binding domain (LssCWT), binding the cell wall of bacteria comprising an amino acid sequence shown in SEQ ID NO: 2 or having at least 80% identity thereto, and
wherein the recombinant polypeptide optionally has a peptide linker linking the LytM catalytic domain and the lysostaphin cell wall binding domain,
wherein the recombinant polypeptide exhibits bacteriolytic activity in raw milk and/or serum;
wherein the recombinant polypeptide is for in-vivo treatment of mastitis or prevention of developing mastitis in the female human or non-human animal, comprising cattle, domesticated and wild bovines, cow, goat, sheep, giraffe, deer, gazelle, antelope, sows, mares, cats, dogs;
wherein the recombinant polypeptide is administered to a mammary gland, lactiferous canal, lactiferous ducts, nipple, teat, udder; and exhibits bacteriolytic activity in raw milk.

36. (canceled)

37. The method according to claim 35, wherein the recombinant polypeptide is administered to raw milk in udder of by injection, infusion, rinsing or dipping.

38-54. (canceled)

55. The recombinant polypeptide for use according to claim 1, wherein the signal sequence is selected from SEQ ID NO: 9-19.

56. The recombinant polypeptide for use according to claim 1, wherein bacterium of the genus Staphylococcus is Staphylococcus aureus.
